(19) United States
(12) Patent Application Publication
(10) Pub. No.: US 2022/0401674 A1
(43) Pub. Date: Dec. 22, 2022

(54) VENTILATION DEVICE, PROCESS, COMPUTER PROGRAM AND DEVICE FOR DETERMINING AN INDICATOR OF AN INTRINSIC END-EXPIRATORY PRESSURE

(71) Applicant: Drägerwerk AG & Co. KGaA, Lübeck (DE)

(72) Inventor: Marcus EGER, Lübeck (DE)

(21) Appl. No.: 17/843,547

(22) Filed: Jun. 17, 2022

(30) Foreign Application Priority Data
Jun. 18, 2021 (DE) 10 2021 115 867.2

(51) Int. Cl. A61M 16/00

(52) U.S. Cl. CPC A61M 16/024; A61M 16/0003; A61M 2016/0027; A61M 2230/42; A61M 2230/60; A61M 2016/0033

(57) ABSTRACT

A process and a device determine an indicator of an intrinsic end-expiratory pressure in the lungs of a patient. Embodiments are based on the device, ventilator with the device, and the process using the device that includes an interface arrangement configured for an exchange of information with a ventilation device and a control unit that determines first information on a first breathing pressure generated by muscles of the patient, at a first time, at which an inhalation attempt of the patient is present and determines second information on a second breathing pressure generated by the muscles of the patient, at a second time, at which breathing gas flow towards the patient starts. The control unit further determines the indicator of the intrinsic end-expiratory pressure based on the first information and based on the second information.

### CROSS REFERENCE TO RELATED APPLICATIONS

This application claims the benefit of priority under 35 U.S.C. § 119 of German Application 10 2021 115 867.2, filed Jun. 18, 2021, the entire contents of which are incorporated herein by reference.

### TECHNICAL FIELD

The present invention pertains to a ventilation device, to a process, to a computer program and to a device for determining an indicator of an intrinsic end-expiratory pressure in the lungs of a patient, and especially, but not exclusively, to a concept for the determination of the intrinsic end-expiratory pressure, iPEEP, on the basis of an analysis of a time course (time curve) of a breathing pressure generated by the muscles of the patient.

### TECHNICAL BACKGROUND

The maintenance and recovery of the spontaneous breathing for the patient had a high priority for a long time in intensive care. If spontaneous breathing is not possible for the patient, a lung-protective ventilation is used, which shall damage the lung tissue as little as possible. The protection of the respiratory muscles, especially of the diaphragm, has only recently moved into the focus.

Details on the background and the state of the art of the present invention are found, for example, in the following documents (which are hereby incorporated by reference): U.S. Pat. No. 5,820,560, WO 2019154834 A1, WO 2019154837 A1, WO 2019154839 A1, WO 2020079266 A1, WO2020188069 A1, DE 10 2019 006 480 A1, US 20170252558 A1, US20150366480 A1, US20120103334 A1, DE 102019007717 B3, US2021205561 A1, DE 10 2007 062 214 B3, WO 2018143844 A1, DE 10 2015 011 390 A1, US 2022 072249 A1, and US2009114224 A1.

Reference should, furthermore, be made to the publications listed below (which is hereby incorporated by reference) in connection with the background of the present invention concerning a concept for determining an indicator of an intrinsic end-expiratory pressure (iPEEP) in the lungs of a patient on the basis of an analysis of a time course of a breathing pressure generated by the muscles of the patient. The sources mentioned below provide additional information on the technical, medical engineering, medical as well as clinical background. The following list comprises—in an incomplete form—an exemplary selection of documents and publications on the detection and the processing of different measured signals or signals in/at the human body and the utilization thereof within the framework of a ventilation, a ventilation control, as well as in the field of ventilation with breathing stimulation.
Walker, D. J.: “Prediction of the Esophageal Pressure by the Mouth Closing Pressure after Magnetic Stimulation of the Phrenic Verve,” Dissertation, Freiburg University: 2006.
Kahl, L. et. al.: “Comparison of Algorithms to Quantify Muscle Fatigue in Upper Limb Muscles Based on sEMG Signals,” Medical Engineering & Physics: 2016.
Jansen D. et. al.: “Estimation of the Diaphragm Neuromuscular Efficiency Index in Mechanically Ventilated Critically Ill Patients,” Critical Care: 2018.
Liu L. et. al.: “Neuroventilatory Efficiency and Extubation Readiness in Critically Ill Patients,” Critical Care: 2012.
Cattapan, S. E. et. al: “Can Diaphragmatic Contractility be Assessed by Airway Twitch Pressure in Mechanically Ventilated Patients?,” Thorax: 2003.
Younes, M. et. al.: “A Method for Monitoring and Improving Patient Ventilator interaction,” Intensive Care Med: 2007.
Blanch Lluis et. al.: “Measurement of Air Trapping, Intrinsic end-Expiratory Pressure, and Dynamic Hyperinflation in Mechanically Ventilated Patients,” Respiratory Care: 2005.
Purro A. et. al.: “Static Intrinsic PEEP in COPD Patients during Spontaneous Breathing,” AJRCCM: 1988.
Bernardi E. et. al.: “A New Ultrasound Method for Estimating Dynamic Intrinsic Positive Airway Pressure: A Prospective Clinical Trial,” AJRCCCM 2018.
Pisani L. et. al.: “Noninvasive Detection of Positive End-Expiratory Pressure in COPD Patients Recovering from Acute Respiratory Failure,” European Respiratory Journal: 2016.
Bellani, G. et. al.: “Clinical Assessment of Auto-positive End-expiratory Pressure by Diaphragmatic Electrical Activity During Pressure Support and Neurally Adjusted Ventilatory Assist.” Anesthesiology: 2014.
Younes, M.: “Dynamic Intrinsic PEEP (PEEPi,dyn): Is It Worth Saving?”, AJRCCM: 2000.
Shalaby, R. E.-S.: “Development of an Electromyography Detection System for the Control of Functional Electrical Stimulation in Neurological Rehabilitation,” Dissertation, 2011.
BELLANI, G. ET AL.: “The Ratio of Inspiratory Pressure Over Electrical Activity of the Diaphragm Remains Stable During ICU Stay and Is Not Related to Clinical Outcome” RESPIRATORY CARE; APRIL 2016; VOL 61 NO 4.
Putensen, C. et al: “Long-Term Effects of Spontaneous Breathing During Ventilatory Support in Patients with Acute Lung Injury,” AM Journal Respir. Crit. Care Med.,” 2001.
Otis, A. B. et al.: “Mechanics of Breathing in Man,” J. Appl. Physiol., 1950.
Osuagwu, B. A. C. et al.: “Active Proportional Electromyogram Controlled Functional Electrical Stimulation System, Nature, 2020.
Virtanen, J. et al.: “Instrumentation for the Measurement of Electric Brain Responses to Transcranial Magnetic Stimulation,” Med. Biol. Eng. Comput., 1999.
   Reference will be made at times to these and other documents in connection with special aspects of the present invention in the course of the description.

To avoid a lack of clarity based on linguistic wordings, some references should be made at the beginning of the application for the understanding as well as some explanations shall be given concerning the use of terms. In the description and/or in the patent claims, the wordings in the verbal form, nominalized verbal form, which are used in the process and also due to embodiments of the control unit in embodiments of the devices according to the present invention, for example and especially “a determining,” “a determining,” “a stimulating,” “a performing,” “an outputting,” “a detecting” are used within the framework of the present invention or inventions with the same meaning as wordings with nouns in the nominalized form, for example and especially “a determination,” “a determination,” “a performance,” “an output,” “a detection,” “a stimulation.” Equivalent and similarly acting meanings are obtained in respect to the disclosure of the invention or inventions for the wordings with nouns, nouns, nominalized verbs and verbs, so that reference is or can also always mutually be made for features and details between the verbal form and the nominalized form concerning the disclosure. Wordings with “performances of determinations, determinations, detections, detections, inputs, outputs, etc.” shall also be considered to be included in the equivalent and similarly acting meanings.

The respiratory muscles comprise the main muscle acting during inhalation, the diaphragm, and the auxiliary muscles. These include, among other things, the external intercostal muscles (acting during inhalation) and the internal intercostal muscles (acting during exhalation) and the abdominal muscles acting during exhalation. It was thus determined that the diaphragm becomes atrophied due to long ventilation times and excessive assistance of the spontaneous breathing, and this requires an extensive weaning. On the other hand, the respiratory muscles may be exhausted and damaged (fatigue) because of increased respiratory load (obstruction, restriction). Some patients tend to make excessive intrinsic breathing efforts in certain situations, which may in turn damage the lungs. Novel systems and processes, by means of which the respiratory muscles can be stimulated, have recently been published—as listed concerning the background of the present invention. All muscles can be stimulated directly by activation of the muscle fibers or of the supplying efferent nerves. For example, the muscle fibers of the diaphragm can be stimulated directly transcutaneously. As an alternative, the phrenic nerve, which is responsible for the contraction of the diaphragm, can be stimulated. Activation of the muscles and contraction take place in both cases. The goal of these processes is to improve the weaning, to promote the removal of secretion and possibly also to avoid ventilation or breathing assistance. Unlike during ventilation or assisted spontaneous breathing, administration of a breathing gas is not necessary in this case. As described in connection with the background of the present invention, a flow and pressure sensor can be used to determine the work of breathing of the patient and to adapt the stimulation such that a time corridor is obtained. However, there is no information technological connection to a ventilator. Parameters of the mechanics of breathing must be taken from the graphic user interface of the ventilator and be entered manually into the separate stimulator. It is, furthermore, not possible to rely on the indicated values of the mechanics of breathing, which are calculated from pneumatic signals, in conventional ventilators as long as the patient is breathing spontaneously to a great degree, i.e., the determined indicator of the work of breathing is only a rough estimate. Accordingly, there is no method so far that can adequately adjust and coordinate the ventilation and the stimulation especially in view to the work of breathing to be performed. As described in connection with the background of the present invention, it is self-evident and known that stimulation and ventilation must be coordinated in their basic mechanism. However, there is no method so far that can predefine the degree of assistance and of stimulation, e.g., depending on a therapy goal. The minute volume necessary for the patient as well as threshold values and limits of the mechanical pressure assistance (triggers, pressures, frequencies) are usually set. The component to be contributed by the patient can hardly be set, because there is no sufficiently precise possibility so far for splitting the work of breathing between machine and patient. Manually coordinated processes, in which mechanics of breathing parameters of the ventilator used are transferred manually and are then used in the stimulator, are practically unaffordable: The parameters change, e.g., after repositioning of the patient. Consequently, they would have to be entered repeatedly. In addition, they are very inaccurate as long as only pneumatic signals were used in the ventilator for determining the parameters. Consequently, the actual contribution of the patient is hardly known, since it would be necessary for this to know the contribution of the patient to the driving pressure (Pmus) or breathing gas flow (FlowMus).

DE 102019006480 describes, for example, a process, which makes possible a separate estimation of these work of breathing components by means of electromyography of the respiratory muscles.

Electromyography (EMG) is a neurological examination for living beings, in which the natural electrical activity of a muscle is measured. Electromyography (EMG) makes it possible to determine the force with which a muscle is exerted. Measurements on superficial muscles are also called sEMG. Electrical Impedance Myography (EIM) is a non-invasive technique for assessing the health of the muscles, wherein the properties of individual muscles or muscle groups or even the composition of muscles and the microscopic structures of muscles can also be examined by means of electrical impedance measurements. Myomechanography (MMG) is a process for detecting elastic, viscous and plastic qualities of muscles. The parameter Pmus represents a variable derived from an EMG signal detected by measurement (electromyogram), from an sEMG signal (Surface Electro-Myogram), from an EIM signal (Electrical Impedance Myogram) or from an MMG signal (mechanomyogram). The parameter Pmus indicates here a pressure level, which has been elicited on the basis of a muscle breathing effort of a patient. The cause of the muscle breathing effort may be initiated by the patient themself in the form of a spontaneous breathing activity and/or it may have been elicited by means of an external, for example, electrical, magnetic or electromagnetic stimulation. The muscle breathing effort may be derived, on the one hand, indirectly from electrical, electromagnetic or magnetic signals. Muscle breathing efforts may also be detected directly by measurement as a pressure difference against a reference pressure, for example, by means of a pressure measurement at the thorax of a patient. The ambient pressure may be selected here as a reference pressure or it is also possible to select a pressure level, which is provided by a ventilator. Pressure levels typically provided by ventilators are, for example, an inspiratory pressure level, usually designated as an inspiratory pressure or inspiration pressure Pinsp, as well as, for example, an expiratory pressure level, usually called an expiratory pressure or expiration pressure Pexp; the so-called PEEP (positive end-expiratory pressure), which describes a pressure level which can be detected by measurement at the end of the exhalation in the airways of the patient as a pressure difference against the ambient pressure, represents a special case of an expiratory pressure level. Both the inspiratory pressure Pinsp and the expiratory pressure Pexp are detected as a pressure difference against the ambient pressure and are usually stated in the unit mbar. Such a parameter Pmus may also be called respiratory muscle pressure Pmus. The terms “muscle airway pressure,” “respiratory muscle pressure” are used in the description and/or in the patent claims within the framework of the present invention or inventions with wordings such as “parameter Pmus,” “pressure parameter Pmus,” “pressure parameter or parameter P, Pmus, which indicates a pressure which has been caused by a muscle breathing effort of a patient,” “a breathing pressure Pmus generated by the muscles of a patient” as terms having the same meaning and producing the identical effect, so that reference can mutually be made concerning the term.

The parameter Flowmus represents a variable derived from the parameter Pmus. Higher frequencies in the signal curve of the parameter Pmus can provide an indicator of muscle-related components, flow direction and reversal of the flow direction of the airway flow. The high-pass filtering of the parameter Pmus can be used to determine the parameter Flowmus. Flow rates that flow into the patient as volumes during inhalation phases, i.e., which are inhaled, or which flow out of the patient during exhalation phases, i.e., which are exhaled, are designated by the term airway flow. The parameter Flowmus indicates here a flow rate with a flow direction, and the cause of the flow is based on a muscle breathing effort of a patient. The cause of the muscle breathing effort may be initiated by the patient themself in the form of a spontaneous breathing activity and/or elicited by means of an external, for example, electrical, magnetic or electromagnetic stimulation. Such a parameter Flowmus may also be called a muscle airway flow or even as a respiratory muscle flow Flowmus. The signal processing with high-pass signal filtering of the signal curve of the parameter Pmus makes it possible to determine and find muscle-related changes in the phases of breathing and in times in the parameter Flowmus, at which a reversal of the sign of the parameter Flowmus has taken place. The reversal of the sign of the parameter Flowmus indicates here times at which a change of the breathing phases between inhalation phases (inhalation) and exhalation phases (exhalation), which are based on muscle breathing efforts of the patient, take place. The terms “muscle airway flow,” “respiratory muscle flow” with wordings such as “parameter Flowmus,” “flow parameter Flowmus,” “flow parameter or parameter Flow, Flowmus, which indicates a pressure which has been caused by a muscle breathing effort of a patient,” and “a flow rate Flowmus brought about by the muscles of a patient” are used in an equivalent manner or in a manner producing the identical effect in the description and/or in the patent claims within the framework of the present invention or inventions, so that reference can mutually be made concerning the term. The following list is used to clarify some terms used within the framework of this application:
An airway pressure is defined in the sense of the present invention as a pressure or as a pressure level—mostly and usually above the ambient pressure—in the airways, in the lungs, in the trachea of a living being.
A muscle airway pressure or a respiratory muscle pressure is defined in the sense of the present invention as a breathing pressure brought about by muscles, as a component of the airway pressure, especially on the basis of—spontaneous or stimulated—muscle activity of the respiratory muscles and/or muscle activity of the auxiliary respiratory muscles of the living being.
An airway flow or a breathing gas flow is defined in the sense of the present invention as any movement of quantities of breathing gas as a quantity of inhaled gases to and into a living being, as well as out of and from a living being as a quantity of exhaled gases.
A muscle airway flow (or a respiratory muscle flow) is defined in the sense of the present invention as any movement of quantities of breathing gas as a quantity of inhaled gases to and into a living being, as well as out of and from a living being as a quantity of exhaled gases on the basis of—spontaneous or stimulated—muscle activity of the respiratory muscles and/or of muscle activity of the auxiliary respiratory muscles of the living being.

The terms muscle airway pressure and respiratory muscle pressure are used synonymously within the framework of the present invention.

The terms muscle airway flow and respiratory muscle flow are used synonymously within the framework of the present invention.

The electrical activity of the diaphragm (EAdi) is recorded by means of a modified gastric probe equipped with electrodes in order to control the pressure assistance of the ventilator proportionally to this electrical activity in processes with “Neurally Adjusted Ventilatory Assist” (NAVA), as described, e.g., in: Sinderby et al.: “Is One Fixed Level of Assist Sufficient to Mechanically Ventilate Spontaneously Breathing Patients?,” Yearbook of Intensive Care and Emergency Medicine, 2007, as well as in Sinderby et al.: “Neural Control of Mechanical Ventilation in Respiratory Failure,” Nature Medicine, 1999.

An especially proportionally assisting NAVA process with the use of a signal for the electrical activity of the diaphragm, whose peculiar feature is that the electrical activity of the diaphragm, which is needed for a defined tidal volume (the so-called neuroventilatory efficiency) shall be maintained at a constant value by means of a “closed-loop” control, is known from U.S. Pat. No. 7,021,310 B1.

Variants of how, for example, the pressure parameter Pmus or the “muscle airway pressure” or respiratory muscle pressure Pmus, pₘᵤₛ(t) can, for example, be determined arise from U.S. Pat. No. 2009 159 082 AA (which is hereby incorporated by reference) and DE 10 2007 062 214 B3 (which is hereby incorporated by reference), to the explanations of which reference shall also expressly made in the description of this application concerning the disclosure concerning terms such as “muscles,” “respiratory muscles,” “muscle airway pressure,” “respiratory muscle pressure,” “parameter Pmus,” “pressure parameter Pmus,” “pressure parameter or parameter Pmus, which indicates a pressure that has been caused by a muscle breathing effort of a patient.”

The respiratory muscle pressure pₘᵤₛ(t) can be determined, for example, in the following manner:
a) Calculation from measured values for airway pressure, volume flow Flow(t), from which the tidal volume Vol(t) is also obtained by integration, as well as from the lung mechanical parameters R (resistance) and E (elastance).
b) Determination by equating with the negative airway pressure −Pₒₖₖ(t) measured during an occlusion, wherein the lung mechanical parameters R and E are either also calculated or are predefined.
c) Determination by means of an esophageal catheter, which is equipped with pressure sensors for measuring the intrathoracic pressure Pₑₛ). The esophageal catheter may optionally be configured and used for measuring the abdominal pressure P_{abd}(t).
d) Determination by means of an array of surface electrodes or sensors, which, arranged on the thorax, provide electromyographically or mechanomyographically determined electrical signals, which can correspondingly also be related to the respiratory muscle pressure Pₘᵤₛ(t) by means of suitable assignment rules, tables, functions or conversion parameters, for example, the so-called “neuromechanical efficiency,” or “neuromuscular efficiency” (NME) and correspondingly also with a volume Vₘᵤₛ(t) by means of the so-called “neuroventilatory efficiency” (NVE).
e) Determination by means of a gastric probe equipped with electrodes, which yield electrical signals, which can be related to the respiratory muscle pressure Pₘᵤₛ(t) by means of suitable assignment rules, tables, functions or conversion parameters.

The variants of the determination of Pₘᵤₛ(t) and Pmus listed in a) through e) and the additional components, such as sensors, electrodes, surface electrodes, pressure sensors, flow sensors, gastric probe, esophageal catheter, which are necessary for the determination of these variables in a device, in a system or in a process, are obtained corresponding to the variant. The breathing activity signal u_{emg}(t) can be subjected to a transformation into a pressure signal p_{emg}(u_{emg}(t)) by means of a predefined transformation rule. The transformation rule can be determined by linear or nonlinear regression between u_{emg}(t) and pₘᵤₛ(t) or also with other procedures, e.g., with neuronal networks, machine learning, or simple scaling. For example, the following linear regression equation

pₘᵤₛ₌₍ₜ₎₌ₐ₀+aᵢ*u_{emg}(t)+a₂*u²_{emg}(t)+a3*u³_{emg}*(t)+(t)

can be used to determine the regression coefficients for the transformation rule being sought, with which a transformed p_{emg}(t) signal is ultimately obtained for the further use, for example, for the ventilation control and/or for the stimulation.

The so-called “intrinsic PEEP” or Auto PEEP (iPEEP) is the driving pressure, which is necessary to exhale at the end of the exhalation within the framework of Dynamic Hyperinflation-based volume (so-called “trapped volume”). Damaged lung mechanics (e.g., expiratory airway flow limitation), impaired spontaneous breathing or incorrect setting of the mechanical ventilation are considered to be causes. The iPEEP is thus an important diagnostic parameter for patients with obstructions, e.g., as a result of COPD (chronic obstructive pulmonary disease). An attempt is made in the therapy to reduce the iPEEP itself or the action thereof (e.g., by adaptation of the external PEEP or CPAP (continuous positive airway pressure) levels). The dynamic hyperinflation and overinflation shall thus be reduced. An increase of the work of breathing is prevented due to the shifting of the working point of the pressure-volume curve into the linear area of the compliance of the lungs. At the same time, the muscle fibers of the diaphragm can contract more efficiently. The advantage can thus finally be achieved to counteract an impending exhaustion of spontaneously breathing patients. There are usually two scenarios for the determination of the iPEEP. The (static) iPEEP is determined by means of end-expiratory occlusion in passively ventilated patients, wherein the subsequent rise in pressure is detected. The rise in pressure corresponds to the pressure, which the “trapped” volume not exhaled exerts when the respiratory muscles or chest wall are/is relaxed. Conventional ventilators have a function for end-expiratory occlusions, so that the measurement of the iPEEP is possible. However, a manual intervention (namely the resolution of the occlusion) is necessary. Furthermore, the ventilation can be easily adapted, e.g., by changing the I:E ratio (ratio of inhalation time to exhalation time) to reduce the iPEEP. Spontaneously breathing patients, and especially patients with COPD, suffer considerably from iPEEP. It hinders spontaneous breathing, since a considerable breathing effort is necessary because of the prestressing (recoil, restoring force) in order to stop the expiratory flow, i.e., “Flow” at all (to become zero); however, the exceeding of the zero line is necessary for the usual pneumatic triggering of breaths. However, spontaneously breathing patients usually barely tolerate end-expiratory occlusions; the occlusion must be carried out for a sufficiently long time until the patient relaxes his muscles, and especially for valid results. This may not be possible in all situations. An administration of sedatives only for the purpose of the determination of the iPEEP can be justified only in rare cases. In addition, the reduction of the iPEEP after a single measurement is not so simple to achieve, since the respiratory drive and the breathing rhythm of the patient must be taken into consideration, i.e., the occlusion maneuver would have to be repeated. In case of spontaneously breathing patients, the (dynamic) iPEEP can be better determined by means of difference formation of the esophageal pressure at two times:
The first time is the beginning of the inhalation effort,
the second time is the moment, in which the flow passes through the zero line.

However, this measurement requires the (invasive and difficult) insertion of an esophageal pressure catheter, which normally cannot be harmonized with routine clinical practice. Meaningful values of the esophageal pressure can be measured only when the catheter is placed correctly. The processes described are drawbacks for spontaneously breathing patients since either end-expiratory occlusions must be carried out possibly repeatedly in conjunction with sedation or an invasive and difficult measurement of the esophageal pressure is necessary.

So-called p0.1 maneuvers in conjunction with a diagnostic ultrasound for avoiding an invasive esophageal pressure measurement are known from the state of the art.

See also, for example, Bernardi, E. et al.: “A New Ultrasound Method for Estimating Dynamic Intrinsic Positive Airway Pressure: A Prospective Clinical Trial,” AJRCCM, 2018, as well as Pisani, L. et al.: “Noninvasive Detection of Positive End-Expiratory Pressure in COPD Patients Recovering from Acute Respiratory Failure,” European Respiratory Journal, 2016 for additional special features for using diagnostic ultrasound. Such an ultrasound method requires, however, a lot of experience and is often not available at bedside.

The document Bellani, G. et al.: “Clinical Assessment of Auto-positive End-expiratory Pressure by Diaphragmatic Electrical Activity During Pressure Support and Neurally Adjusted Ventilatory Assist,” Anesthesiology, 2014, describes a process within the framework of a clinical study. The electrical activity signal EAdi of the crural flaps of the diaphragm, which is detected invasively via an esophageal catheter (Maquet NAVA) equipped with electrodes, is used here. The EAdi signal is read and multiplied by a factor at the beginning of the inspiratory flow for the determination of the Dynamic intrinsic PEEP. The need for an invasive esophageal catheter for deriving the activity signal EAdi and the carrying out of uncomfortable end-expiratory occlusions for the determination of the factor are drawbacks.

The following references will be used below, and the following variables are variable over time, as this is indicated in Table 1 below:

TABLE 1

| | |
|---|---|
| Vol_{ges}, | The total volume or the total volume flow, which flows from |
| Vol′_{ges} | the ventilator to the patient or in the opposite direction |
| Volₘᵤₛ, | The volume or the volume flow which the patient achieves |
| Vol′ₘᵤₛ | with his respiratory muscles, usually not measurable directly |
| Volᵥₑₙₜ, | The volume or the volume flow which the ventilator achieves |
| Vol′ᵥₑₙₜ | |
| P_{aw} | The pressure present at the ventilator |
| P_{drv} | The total pressure acting on the lungs (driving pressure) |
| Pᵥₑₙₜ | The pressure generated by the ventilator |
| Pₘᵤₛ | The pressure which the patient generates with his respiratory |
| | muscles |
| R, E | Resistance and elasticity of the respiratory system, comprising |
| | the lungs and the chest |
| PEEP | End-expiratory pressure |
| iPEEP | Intrinsic end-expiratory pressure, which remains in the lungs |
| | after a breath |
| τ, tau | Respiratory time constant of the respiratory system, which is |
| | considered to be a linear system |
| s | The frequency (complex number) |

### SUMMARY

Based on this, one object of the present invention is to provide an improved concept for determining an indicator of an intrinsic end-expiratory pressure in the lungs of a patient.

The object is accomplished according to a device for determining an indicator of an intrinsic end-expiratory pressure in lungs of a patient according to the invention. The object is accomplished according to a ventilation device with a device for determining an indicator of an intrinsic end-expiratory pressure in lungs of a patient according to the invention. The object is accomplished according to a process according to the invention and a computer program (computer readable media—software provided on a non-transient, tangible medium (or media)) with a program code for carrying out all or some of the process when the program code is executed on a computer, on a processor or on a programmable hardware component.

Advantageous embodiments of the present invention appear from this disclosure and are explained in more detail in the following description partially with reference to the figures.

Exemplary embodiments on the basis of the core idea that the above-mentioned drawbacks can be avoided when a non-invasive, continuous Pmus estimation is used instead of the invasive measurement of the esophageal pressure or of the electrical activity of the diaphragm. As described in DE 10 2007 062 214 B3, Pmus can be continuously estimated, for example, in conjunction with the measurement of the respiratory surface electromyogram (sEMG). The esophageal pressure, Pes, and the respiratory muscle pressure, Pmus, are linked together via the equation

Pes=Ecw·V−Pmus (1)

(Ecw is the restoring force/elasticity of the chest wall). The accumulated volume is still zero at the start of a breath, so that Pes and Pmus are identical (except for the sign). Now, instead of using the esophageal pressure for the difference formation, the estimated Pmus can be used. For example, the beginning of the breathing effort (tA) and the moment, at which the Flow passes through the zero line (tB), are selected as times. The latter is known (for example, in the ventilator as well). E.g., the zero crossing of the component of the flow signal, which stems from the spontaneous breathing, V′mus, can be used to determine the time for the beginning of the breathing effort. It can, as an alternative, be determined by means of a freely selectable breathing effort signal (e.g., of the Pmus) passing through a threshold value.

The intrinsic PEEP is thus obtained, for example, from

iPEEP=ΔPmus=Pmus(tB)−Pmus(tA) (2)

Since a repetition of this measurement and calculation is not a drawback by contrast to carrying out longer occlusions, an improved value of the iPEEP can be obtained by averaging more than one measurement, possibly in case of a plurality of breaths. Exemplary embodiments therefore create a process for determining an indicator of an intrinsic end-expiratory pressure, iPEEP, in the lungs of a patient. The process comprises a determining or a determination of a first piece of information (first information) via a first breathing pressure generated by the muscles of the patient, Pmus (tA), at a first time, tA, at which an inhalation attempt of the patient is present or is beginning. The process further comprises a determination of a second information (second information) on a second breathing pressure generated by the muscles of the patient, Pmus (tB), at a second time, tB, at which a breathing gas flow towards the patient starts. The process comprises, in addition, a determining of the indicator or a determination of the indicator of the iPEEP on the basis of the first information and on the basis of the second information. Exemplary embodiments can thus make it possible to determine the iPEEP from the breathing pressure generated. The determination may comprise a determination of an indicator of a difference or a weighted difference between Pmus (tA) and Pmus (tB). Thus, an efficient basis can be provided for the determination of the iPEEP. In addition or as an alternative, the determination may comprise a determination of an indicator of a quotient or a weighted quotient between Pmus (tA) and Pmus (tB). The consideration of a quotient may contribute to an effective determination of the iPEEP as well. The determination may, in addition, comprise an averaging, a sorting and/or an assessment of measured pressure values from a plurality of breathing phases, for example, in order to make an estimation of the respiratory muscle pressure more robust.

In further exemplary embodiments, the determination of the first information and of the second information may comprise an estimation of the breathing pressure generated by the muscles of the patient on the basis of an electromyographic signal. An electromyographic signal may form a robust and non-invasively detected basis for the estimation of the respiratory muscle pressure.

For example, the determination of the first information and the second information may comprise an estimation of the breathing pressure generated by the muscles of the patient on the basis of an airway pressure generated at the patient Paw, of a tidal volume V and of a tidal volume flow V′. These variables may contribute to an effective or robust estimation and, for example, may be detected pneumatically.

In some exemplary embodiments, the process may also comprise a receiving of information on the generated airway pressure Paw, the tidal volume V and the tidal volume flow V′ from a ventilation system ventilating the patient. Pneumatic signals or pneumatically detected signals (pressure sensors) may thus also be included in the estimation.

The determination of the first information may comprise an estimation of the first time, tA, on the basis of a start of a breathing gas flow in the direction of the patient brought about by the muscles of the patient. The curve or the start of the breathing gas flow may form an effective basis for the estimation of the first time.

For example, the determination of the first information may comprise an estimation of the first time, tA, on the basis of a breathing effort signal of the patient passing through a threshold value. A passing through a threshold value may be implemented and detected in a cost-effective manner.

The determination of the first information may also comprise an estimation of the first time, tA, on the basis of a starting of a spontaneous breathing of the patient. A starting of the spontaneous breathing may be determined, for example, from an activation signal of the respiratory muscles.

In addition or as an alternative, the determination of the second information may comprise an estimation of the second time on the basis of a start of a breathing gas flow in the direction of the patient. The curve or the start of the breathing gas curve is also an effective indicator or effective basis for the estimation of the second time, tB.

The process may comprise, in addition, an averaging, a smoothing, a suppression of outliers or a determination of a median of a plurality of indicators of the IPEEP determined sequentially in time to obtain an improved indicator of the iPEEP. Exemplary embodiments may thus determine a more reliable indicator of the iPEEP.

In further exemplary embodiments, the process may comprise a calibration of the determination of the indicator of the iPEEP on the basis of a measurement of a ventilation device during an occlusion. A calibration may contribute to a determination of a more reliable indicator of the iPEEP.

A further exemplary embodiment is a computer program with a program code for carrying out one of the processes described here when the program code is executed on a computer, on a processor or on a programmable hardware component.

Exemplary embodiments provide, in addition, a device for a ventilation device and for determining an indicator of an intrinsic end-expiratory pressure, iPEEP, in the lungs of a patient. The device comprises one or more interfaces, which are configured for the exchange of information with the ventilation device. The device comprises, in addition, a control unit, which is configured for determining a first information on a first breathing pressure generated by the muscles of the patient, Pmus (tA) at a first time, tA, at which an inhalation attempt of the patient is present or is beginning. The control unit is configured for determining a second information on a second breathing pressure generated by the muscles of the patient, Pmus (tB), at a second time, tB, at which a breathing gas flow towards the patient starts. In addition, the control unit is configured for determining the indicator of the iPEEP on the basis of the first information and on the basis of the second information. Exemplary embodiments may thus also provide a device for the determination of the iPEEP from the generated breathing pressure.

In further exemplary embodiments, the device may comprise one or more sensors for the detection of measured values during a ventilation of a patient. For example, the device may be configured for the detection of measured pressure values or measured pressure signals during the ventilation of a patient.

In exemplary embodiments, the control unit may be configured for carrying out one of the already explained processes or of process steps. The determination may thus comprise a determination of an indicator of a difference or of a weighted difference between Pmus (tA) and Pmus (tB). In addition or as an alternative, the determination may comprise a determination of an indicator of a quotient or of a weighted quotient between Pmus (tA) and Pmus (tB). The determination may also comprise an averaging, a sorting and/or an assessment of measured pressure values from a plurality of breathing phases.

The control unit may be configured to carry out the determination of the first information and of the second information on the basis of an estimation of the breathing pressure generated by the muscles of the patient on the basis of an electromyographic signal. The determination of the first information and of the second information may comprise an estimation of the breathing pressure generated by the muscles of the patient on the basis of an airway pressure generated at the patient Paw, of a tidal volume V and of a tidal volume flow V′.

In further exemplary embodiments, the control unit may be configured to receive information on the generated airway pressure Paw, the tidal volume V and/or the tidal volume flow V′ from a ventilation device currently ventilating the patient.

In some exemplary embodiments, the determination of the first information may comprise an estimation of the first time, tA, on the basis of a starting of a breathing gas flow in the direction of the patient caused by the muscles of the patient. In addition or as an alternative, the determination of the first information may comprise an estimation of the first time, tA, on the basis of a breathing effort signal of the patient passing through a threshold value.

The determination of the first information may comprise an estimation of the first time, tA, on the basis of a starting of spontaneous breathing of the patient. The determination of a second information may comprise an estimation of the second time, tB, on the basis of a starting of a breathing gas flow in the direction of the patient.

In further exemplary embodiments, the control unit may be configured to average a plurality of indicators of the iPEEP determined sequentially in time, to smooth them, to suppress outliers or to determine a median therefrom in order to obtain an improved indicator of the iPEEP. The control unit may be configured to calibrate the determination of the indicator of the iPEEP on the basis of a measurement of the ventilator during an occlusion.

One embodiment according to the present invention shows a device for a ventilation device and for determining an indicator of an intrinsic end-expiratory pressure, iPEEP, in the lungs of a patient, with one or more interfaces, which are configured for the exchange of information with the ventilation device, and with a control unit, which is configured
for a determination of a first information on a first breathing pressure generated by the muscles of the patient, Pmus (tA), at a first time, tA, at which an inhalation attempt of the patient is present,
for a determination of a second information on a second breathing pressure generated by the muscles of the patient, Pmus (tB), at which a breathing gas flow towards the patient starts, and
for a determination of the indicator of the iPEEP on the basis of the first information and on the basis of the second information.

In a preferred embodiment of the device, the device may comprise one or more sensors for detecting measured values during a ventilation of a patient.

In a preferred embodiment, the device may be configured for a detection of measured pressure values or measured pressure signals during a ventilation of a patient.

In a preferred embodiment, the determination may comprise a determination of an indicator
of a difference or of a weighted difference between Pmus (tA) or Pmus (tB), and/or a determination of an indicator
of a quotient or of a weighted quotient between Pmus (tA) and Pmus (tB).

In a preferred embodiment of the device, the determination may comprise an averaging, a sorting and/or an assessment of measured pressure values from a plurality of breathing phases.

In a preferred embodiment of the device, the determination of the first information and of the second information may comprise an estimation of the breathing pressure generated by the muscles of the patient on the basis of
an electromyographic signal,
and/or an estimation of the breathing pressure generated by the muscles of the patient on the basis of an electromyographic signal, and/or on the basis of
an airway pressure generated at the patient Paw, of a tidal volume V and of a tidal volume flow V′.

In a preferred embodiment of the device, the control unit may be configured to receive information on the generated airway pressure Paw, the tidal volume V and the tidal volume flow V′ from the ventilation device ventilating the patient.

In a preferred embodiment of the device, the determination of the first information may comprise an estimation of the first time on the basis of a starting of a breathing gas flow in the direction of the patient caused by the muscles of the patient.

In a preferred embodiment of the device, the determination of the first information may comprise an estimation of the first time
on the basis of a breathing effort signal of the patient passing through a threshold value, and/or an estimation of the first time
on the basis of a starting of a spontaneous breathing of the patient.

In a preferred embodiment of the device, the determination of the second information may comprise an estimation of the second time on the basis of a start of a breathing gas flow in the direction of the patient.

In a preferred embodiment of the device, the control unit may be configured to average a plurality of indicators of the iPEEP determined sequentially in time, to smooth them, to suppress outliers or to determine a median therefrom, to obtain an improved indicator of the iPEEP. Outliers are, for example, measured values or values, which have values deviating from one of the population of values or measured values considered over a longer period of observation due to the disturbances superimposed on the measured values. When this deviation is considered to be significant compared to the population of values or measured values, such outliers can be identified and suppressed by means of signal processing or data processing.

The control unit may be configured in one preferred embodiment of the device to calibrate the determination of the indicator of the iPEEP on the basis of a measurement of the ventilation device during an occlusion.

A preferred embodiment may configure a ventilation device with a device on the basis of the above-described embodiments.

A preferred embodiment may be formed by a process for determining an indicator of an intrinsic end-expiratory pressure, iPEEP, in the lungs of a patient
with a determination of a first information via a first breathing pressure generated by the muscles of the patient, Pmus (tA), at a first time, tA, at which an inhalation attempt of the patient is present,
with a determination of a second information via a second breathing pressure generated by the muscles of the patient, Pmus (tB), at a second time, tB, at which a breathing gas flow towards the patient starts, and
with a determination of the indicator of the iPEEP on the basis of the first information and on the basis of the second information.

A preferred embodiment may be formed by a computer program (computer readable media—software provided on a non-transient, tangible medium (or media)) with a program code for carrying out the process when the program code is executed on a computer, on a processor or on a programmable hardware component.

Some examples of the devices and/or process are explained in more detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a flow diagram showing an exemplary embodiment of a process for the determination of an indicator of an intrinsic end-expiratory pressure, iPEEP, in the lungs of a patient;

FIG. 2 is a schematic view of an exemplary embodiment of a device for a ventilation device and for determining an indicator of an intrinsic end-expiratory pressure, iPEEP, in the lungs of a patient, and of an exemplary embodiment of a ventilation device;

FIG. 3 is a schematic view showing a ventilation of a patient and for the detection of an electromyographic signal;

FIG. 4 is a graph showing a time course (time curve) of a pressure generated by a ventilator, a volume flow between the ventilator and a patient, and a curve of intrinsic breathing efforts of a patient;

FIG. 5 is a graph showing a time course of a volume flow Vol′ and a time course of an airway pressure Paw in an exemplary embodiment;

FIG. 6 is a graph showing signal curves of an EMG signal, of a respiratory muscle pressure, of an airway pressure and of a volume flow in an exemplary embodiment;

FIG. 7 is a graph showing signal curves (courses) of an sEMG signal, of a volume flow Vol′ and of a volume flow generated by the muscles of the patient, Vol′mus, in an exemplary embodiment; and

FIG. 8 is a graph showing signal courses (curves) of a volume flow Flow, of an airway pressure Paw, of an esophageal pressure Pes and of a gastric pressure Pga.

### DESCRIPTION OF PREFERRED EMBODIMENTS

Different examples will be described now with detailed reference to the attached figures. The thicknesses of lines, layers and/or areas may be exaggerated in the figures for illustration.

Further examples may cover modifications, equivalents and alternatives, which fall within the scope of the disclosure. Identical or similar reference numbers pertain in the entire description of the figures to identical or similar elements, which may be implemented in a comparison with one another identically or in a modified form, while they provide the same function or a similar function.

It is apparent that if an element is described as being “connected” to or “coupled” with another element, the elements may be connected or coupled directly or via one or more intermediate elements. If two elements A and B are combined with the use of an “or,” this should be understood to be such that all possible combinations are disclosed, i.e., only A, only B as well A and B, unless something else is explicitly or implicitly defined. An alternative wording for the same combinations is “at least one of A and B” or “A and/or B.” The same applies, mutatis mutandis, to combinations of more than two elements.

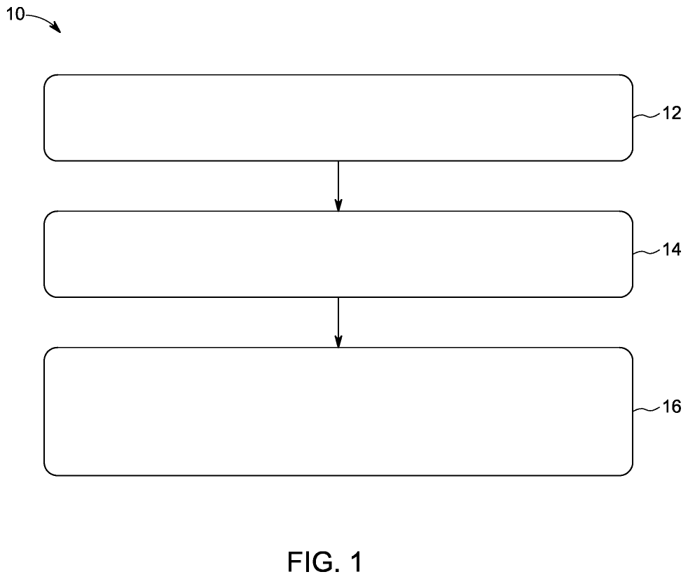

FIG. 1 shows an exemplary embodiment of a process 10 for determining an indicator of an intrinsic end-expiratory pressure, iPEEP in the lungs of a patient. The process 10 comprises a determination 12 of a first information on a first breathing pressure generated by the muscles of the patient, Pmus (tA), at a first time, tA, at which an inhalation attempt of the patient is present or begins. The process further comprises a determination 14 of a second information on a second breathing pressure generated by the muscles of the patient, Pmus (tB), at a second time, tB, at which a breathing gas flow towards the patient starts. The process comprises a determination 16 of the indicator of the iPEEP or a determination of the indicator of the iPEEP on the basis of the first information and on the basis of the second information.

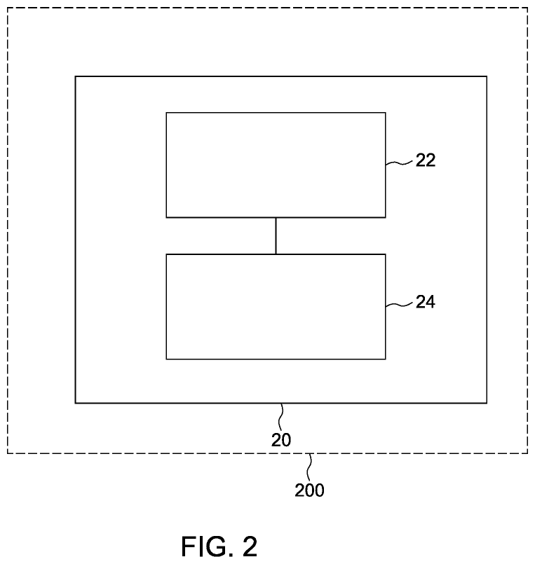

FIG. 2 shows block diagrams of an exemplary embodiment of a device 20 for a ventilation device 200 and for determining an indicator of an intrinsic end-expiratory pressure, iPEEP, in the lungs of a patient, and of an exemplary embodiment of a ventilation device 200. The device 20 comprises one or more interfaces 22, which are configured for the exchange of information with the ventilation device 200 and which are coupled with a control unit 24, which is likewise comprised by the device 20. The control unit 24 is configured to determine 12 a first information on a first breathing pressure generated by the muscles of the patient, Pmus (tA), at a first time, tA, at which an inhalation attempt of the patient is present or begins and to determine 14 a second information on a second breathing pressure generated by the muscles of the patient, Pmus (tB), at a second time, tB, at which a breathing gas flow towards the patient starts. The control unit 24 is configured to determine 16 the indicator of the iPEEP on the basis of the first information and on the basis of the second information. FIG. 2 illustrates, moreover, an exemplary embodiment of a ventilation device 200 with a device 20. In addition, the device 20 may comprise one or more sensors (e.g., pressure sensors, strain sensors, electrical sensors, etc.) for detecting measured values during a ventilation of a patient. For example, the device 20 may be configured to detect measured pressure values or measured pressure signals during a ventilation of a patient.

The device 20 comprises one or more interfaces 22, which are coupled with the control unit 24. The one or more interfaces 22 may be configured, for example, in the form of a machine interface or in the form of a software interface.

The one or more interfaces 22 may be configured in exemplary embodiments as typical interface(s) for communication in networks or between network components or medical devices, e.g., ventilators, sensor units or measuring units, stimulators, etc. For example, they may be configured in exemplary embodiments by corresponding contacts. They may also be configured in exemplary embodiments as separate hardware and comprise a memory (and/or a memory and processor), which at least temporarily stores the signals to be transmitted or the received signals. The one or more interfaces 22 may be configured to receive electrical signals, for example, as a bus interface, as an optical interface, as an Ethernet interface, as a wireless interface, as a field bus interface, etc. They may, moreover, be configured in exemplary embodiments for wireless transmission and comprise a radio front end (wireless receiver/transmitter) as well as corresponding antennas. Input and/or output devices, for example, display screen, keyboard, mouse, may also be connected via the one or more interfaces 22 in order to detect user inputs and/or to make outputs possible.

The control unit 24 may comprise in exemplary embodiments one or more freely selectable controllers, microcontrollers, network processors, processor cores, such as digital signal processor cores (DSPs), programmable hardware components, etc. Exemplary embodiments are not limited here to a particular type of processor core. Any desired processor core or even a plurality of processor cores or microcontrollers are conceivable for implementing a control unit 24. Implementations in integrated form with other devices are also conceivable, for example, in a control unit, which additionally also comprises one or more other functions. A control unit 24 may be embodied in exemplary embodiments by a processor core, a computer processor core (CPU=Central Processing Unit), a graphics processor core (GPU=Graphics Processing Unit), an application-specific integrated circuit core (ASIC=Application-Specific Integrated Circuit), an integrated circuit (IC=Integrated Circuit), a one-chip system core (SOC=System on Chip), a programmable logic element or a field-programmable gate array with a microprocessor (FPGA=Field Programmable Gate Array) as a core of the component or components.

The present description uses the following terms and definitions:

(Muscle) breathing effort:

The term is used in the generic sense. It designates the muscle effort of the patient to generate a respiratory muscle pressure in order to bring about a flow in the airway. If occlusion is performed during the breathing effort, i.e., if the airway flow is interrupted by blockage, no flow develops in the airway, even though a respiratory muscle pressure (which can be measured as “mouth pressure” (pressure in the mouth area) if the airways are open) is generated. There is an isometric contraction of the respiratory muscles in this case. A physiological work always corresponds to the breathing effort, but the physiological work cannot be measured directly. A physical work, which is, by contrast, measurable, is only performed when the contraction is not generated isometrically, i.e., when a flow is generated in the airway.

Breathing assistance:

The breathing assistance is a ventilatory pendant to the muscle breathing effort. The ventilator assists the patient's breathing effort detected (by triggering) synchronously with an assistance stroke. The patient consequently sets the breathing rhythm. It may be a pressure-controlled assistance (the airway pressure is set) or—more rarely—a volume-controlled assistance (the breath volume is set). Physical work is performed by the ventilator in all cases, i.e., a part of the total work of breathing to be performed is taken from the patient.

(Mandatory) ventilation:

The total work of breathing is eliminated from the patient during mandatory ventilation. The breathing rhythm is determined by the machine. The patient is normally passive during the mandatory ventilation, so that there will be no conflict between the human/patient and the machine. The passivity of the patient is often brought about by sedatives and relaxants.

WOB—Work of breathing:

This is the physiological or physical work, which is performed for the breathing or/and ventilation. Even though no physical work is performed in case of isometric contraction in the sense of the kinetic equation, as it will be described below in the further course of the description, other definitions of work, e.g., the so-called pressure-time product (time integral), can be used.

WOBtot—Total work of breathing:

This is the total physiological or physical work, which is performed for the breathing or/and ventilation.

WOBvent—Machine/ventilator-side work of breathing:

This is the component of the work of breathing that is contributed by the ventilator.

WOBmus—Patient-side (muscle) work of breathing:

This is the component of the work of breathing that is performed solely by the patient, both with and without stimulation of the muscles.

WOBspon—Spontaneous (patient-side) work of breathing:

This is the work of breathing performed by spontaneous intrinsic breathing of the patient. The muscles are not stimulated in the process.

WOBstim—Stimulated (patient-side) work of breathing:

This is the work of breathing performed by stimulation of the respiratory muscles of the patient.

Pdrv—Driving pressure:

Pdrv is the sum of the pressures that are generated by the ventilator and by the patient.

Pvent—Ventilation pressure:

Pvent is the pressure that is generated by the ventilator.

Pmus—Muscle pressure:

Pmus is the pressure that is generated by the muscles of the patient alone (the patient's own efforts), both with and without stimulation of the muscles.

Pspon—Spontaneous muscle pressure:

Pspon is the component of the muscle pressure that is generated spontaneously, i.e., without stimulation, by the patient.

Pstim—Stimulated muscle pressure:

Pstim is the component of the muscle pressure that is generated by the patient solely based on the stimulation of the muscles.

WOBbase and PmusBase—Basic breathing load:

The basic breathing load can be equated with the work of breathing or with the driving pressure, which is necessary to overcome the resistive, elastic and optionally other resistances and to reach a sufficient volume (e.g., the minute volume set by the clinical staff) in case of a healthy breathing pattern. The breathing pattern is preferably assumed to be an energy-optimized pattern. The work of breathing or the driving pressure can be generated on the patient side or/and on the machine side. The latter would happen in case of a (mandatory) ventilation.

Breathing load:

The breathing load can be determined by detecting the work of breathing or the driving pressure, which work of breathing or driving pressure is actually generated. The breathing load is normally higher than the basic breathing load, since the breathing rhythm of the patient is not energy-optimized, e.g., the patient seeks to have a higher volume, e.g., based on shortness of breath than is needed or there is an asynchronism between the patient and the ventilator. The ventilator assumes a part of the breathing load in case of breathing assistance.

Activation signal:

This is a signal that detects the neuronal activation of the muscle (caused either by stimulation or by spontaneous breathing effort), e.g., the (s)EMG (surface electromyogram), EIM (electrical impedance myogram), MMG (mechanomyogram). As an alternative, signals that are detected by means of novel optical or acoustic (e.g., ultrasound) technology are also considered. The enveloping curve of the EMG will be used below as an activation signal (designated by “EMG” for simplicity's sake), without excluding other signals. It shall not be ruled out hereby that there are different activation signals of different muscle groups (e.g., diaphragm and intercostal muscles), which yield an activation of their own each. The diaphragm activation signal is in the foreground in the context of the stimulation of the diaphragm (for example, by magnetic stimulation of the phrenic nerve).

### Activatability:

This is the ability electrically to stimulate (activate) the muscles, for example, by electrical or magnetic stimulation. The activation is preferably elicited by a so-called magnetic twitch stimulation, by a transient stimulation pulse with high intensity, which leads to a maximum contraction of the stimulated muscle. Other stimulation patterns would be conceivable as well. The activation may be detected by means of an activation signal, preferably with EMG.

### Efficiency:

The efficiency is a pneumatic target variable (pressure or volume), which is achieved by a muscle activation. The “neuromechanical efficiency” (or “neuromuscular efficiency”) NME relates the muscle pressure produced to the EMG as is described, for example, in WO 2018143844 A1, as well as: Jansen, D. et al.: “Estimation of the diaphragm neuromuscular efficiency index in mechanically ventilated critically ill patients,” Critical Care, 2018. The “neuroventilatory efficiency” NVE relates the volume generated to the EMG as is described, for example, in Liu, L. et al.: “Neuroventilatory efficiency and extubation readiness in critically ill patients,” Critical Care., 2012, 16: R143. The determination of the efficiency often requires maneuvers, e.g., occlusions or changes in the breathing assistance. As is described there, the values have a diagnostic significance, e.g., in the assessment of the progression of the weaning from the ventilator.

Maximum possible breathing effort:

This corresponds to the work of breathing WOBmusMax, to the volume VolMusMax or to the muscle pressure PmusMax, which work of breathing, volume or muscle pressure can be achieved by maximum effort of the respiratory muscles. PmusMax is most likely to be able to be measured in a standardized manner. Thus, the value Pimax is frequently used in the literature as the indicator of the maximum possible muscle pressure, i.e., the maximum pressure produced during mouth closing. Since the voluntary contraction of the diaphragm leads to unreliable events, a so-called (usually magnetic) twitch stimulation, which can be performed independently from the ability of the patient to cooperate, is frequently used recently to elicit the contraction. The PmusMax is normally detected by means of an esophageal catheter, but the mouth closing pressure, which can be measured in a more simple manner, as it is described, for example, in Cattapan, S, E. et al.: “Can diaphragmatic contractility be assessed by airway twitch pressure in mechanically ventilated patients?,” Thorax, 2003; 58:58-62, has likewise proved to be meaningful. Triggering of the stimulation is advantageous here.

### LI Load (Load Index):

This is an indicator that depends on the ratio of the work of breathing produced to the maximum possible work of breathing, but the muscle pressure or another indicator may also be used instead of “work.” The load can be defined for the muscle pressure as

LI=Pmus/PmusMax.

### LBC—Load Bearing Capacity;

This is the ability of the muscles to exert by contraction a defined force or—in case of the respiratory muscles—pressure and thus to be able to perform work. To quantify the load bearing capacity, the muscle pressure/work of breathing produced by the muscles is usually related to the muscle pressure/work of breathing that can be maximally produced. The load bearing capacity can thus be determined quantitatively as a function of the ratio of the basic breathing load to the maximum possible breathing effort. When the basic breathing load exceeds a defined part of the maximum possible breathing effort, the load bearing capacity is no longer present for exclusive spontaneous breathing and ventilation is mandatory. The load bearing capacity can be defined for the muscle pressure as

LBC=1−PmusBase/PmusMax.

### Exhaustion:

This happens after some time when the load bearing capacity of the respiratory muscles is so low that the component contributed by the patient to the breathing load exceeds a defined part of the maximum possible breathing effort, e.g., 50% aa.

Degree of exhaustion:

The degree of exhaustion is linked with the load bearing capacity, with the breathing effort and with the duration hereof, but it cannot be calculated from these exclusively and directly. However, there are measured values, which can be calculated, for example, from the electromyogram of the muscles and can be used as a surrogate for the degree of exhaustion; this is described, for example, in Kahl, L. et al.: “Comparison of algorithms to quantify muscle fatigue in upper limb muscles based on sEMG signals,” September 2016, Medical Engineering & Physics.

The relationships of the relevant variables may also be described on the basis of formulas, some of which will be shown below.

The work of breathing can be calculated as an integral of the corresponding pressure relative to the volume, e.g., for the total work of breathing:

WOBtot=∫Pdrv(t)dV=∫Pdrv(t)·Flow(t)dt.

As an alternative (especially for the case of isometric load), the pressure-time product can be used:

WOBtot˜=∫Pdrv(t)dt.

The driving pressure is divided, analogously to the work of breathing, into different components:

Pdrv=Pvent+Pmus=Pvent+Pspon+Pstim

WOBtot=WOBvent+WOBmus=WOBvent+WOBspon+WOBstim.

The flow or the volume can likewise be divided by calculation into the different components analogously to the work of breathing:

Flow=FlowVent+FlowMus=FlowVent+FlowSpon+FlowStim and

Vol=VolVent+VolMus=VolVent+VolSpon+VolStim.

Pdrv=Pvent+Pmus=R·Flow+E·Vol+const

is valid for the basic kinetic equation at the breathing circuit.

When the flow and the volume are defined (as described above) each as the sum of the components contributed by the ventilator and by the patient,

Pvent=R·FlowVent+E·VolVent+const

and Pmus=R·FlowMus+E·VolMus+const

are obtained for Pvent and Pmus.

The work of breathing of the ventilator and that of the patient can thus be calculated:

WOBvent=∫Pvent·Flow dt=∫Pdrv·FlowVent dt,

WOBmus=∫Pmus·Flow dt=∫Pdrv·FlowMus dt.

That there always are two possibilities can be derived from the kinetic equation (see above) and it can be proved by insertion into the integrals. In a simplified hypothesis, Pmus can be assumed to be proportional to the EMG signal:

Pmus=NME·EMG

or, for example, as a linear combination of the EMG signals of two muscle groups:

Pmus=NME1·EMG1+NME2·EMG2,

wherein NME, NME1 and NME2 represent the neuromechanical efficiency of the respective muscle group. The kinetic equation

Pvent+Pmus=R·Flow+E·Vol+const

thus changes to

Pvent=R·Flow+E·Vol+const−NME EMG.

How NME can be determined is already described and explained by the aspect “efficiency” within the framework of the present invention. The muscle activation is the sum of the spontaneous activity and of the activity triggered by stimulation

EMG=EMGspon+EMGstim,

wherein it is assumed that the amplitude EMGstim\hat{ } of the activity triggered by stimulation is linked with the activatability k and with the amplitude of the stimulation intensity Istim\hat{ } in a multiplicative manner:

EMGstim\hat{ }=k·Istim\hat{ }.

The scalar relationship can then be used when indicators of the stimulation intensity and activation are valid for broader time ranges, e.g., whole breaths. However, the stimulation takes place now typically as a sequence of weighted pulses with a distance of 20-100 msec corresponding to 10-50 Hz (preferably 40-50 msec corresponding to 20-50 Hz). Each individual pulse (twitch) triggers an individual activation, but the breath-like shape of the activation signal is obtained only after the entire pulse sequence, i.e., the time course of the triggered activity EMGstim(t) differs markedly from the time course of the stimulation intensity Istim(t). The activatability can be represented as a simple constant (or characteristic) only in case of time-averaged variables. The kernel-based estimation is possible for the time characteristic, e.g., with the simple hypothesis

EMGstim(t)=Istim(t)*k(t),

in which * is the convolution symbol and k(t) is the core, i.e., the core of the modeling of the activatability, which core is to be estimated. Constant components (offsets) of the activation in the sense of a tonic muscle tension are ignored here. Istim(t) is usually a sequence of transient stimulation pulses. Then, k(t) corresponds to the pulse response of the activation to a stimulation pulse of the intensity Istim(t). There are many methods for estimating the kernel k(t), e.g., methods of system identification, stimulus-dependent averaging (for example, analogously to the peri-stimulus-time histogram) or least-squares estimation method. In the equation

EMG=EMGspon+Istim(t)*k(t),

EMGspon is assumed to be an unwanted signal, which is minimized by adaptation of the kernel. Finally, the spontaneous activity EMGspon can thus be determined as well, so that all factors of the kinetic equation

Pvent(t)=R·Flow(t)+E·Vol(t)+const−NME·[EMGspon(t)+k(t)*Istim(t)]

are known. The components of the total work of breathing and of the driving pressure can thus be determined and used for controlling the ventilation and the stimulation. Instead of estimating the sample values of the kernel, a parametric estimation may be carried out as well. The kernel could thus be considered to be a system pulse response and the parameters thereof could be identified. Correspondingly,

EMG=EMG1+EMG2=EMG1spon+EMG1stim+EMG2spon+EMG2stim

and

Pvent(t)=R·Flow(t)+E·Vol(t)+const−NME1·[EMG1spon(t)+k1(t)*Istim1(t)]−NME2[EMG2spon(t)+k2(t)*Istim2(t)]

are valid for the activation of, e.g., two muscle groups possibly by means of stimulation.

The components of the work of breathing, which are produced by different muscle groups, can thus be determined. The specific stimulation makes possible especially stimulation maneuvers, which specifically affect defined muscle groups and lead to the activation. As a result, an estimation of the neuromechanical efficiencies and of the kernels or stimulation pulse responses is comparatively simple. Instead of using work of breathing (WOB) or muscle pressure (Pmus) as target variables for the stimulation, it would also be possible, as an alternative, to use the component of the flow that is caused by the muscles, FlowMus. FlowMus (flow rate) or its integral over time, VolMus (volume), may be advantageous under some circumstances, as can be comprehended, for example, on the basis of the document US 20170252558 A1. It would be advantageous for this for the clinical staff to be very familiar with the terms flow and volume contrary to muscle pressure or work of breathing. The splitting of the flow or volume into patient and machine components is a basis in exemplary embodiments. When FlowMus is available, VolMus (as a time course), but also VTmus (tidal volume produced by muscles) or MVmus (minute volume contributed by muscles) can be determined in a very simple manner by an integration. These variables may be important for the respiratory diagnostics and therapy.

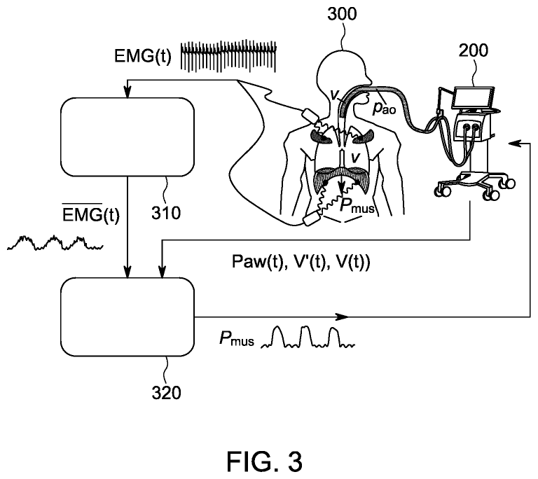

FIG. 3 shows an overview figure of the ventilation of a patient and of the detection of an electromyographic signal. FIG. 3 shows on the right side a ventilation device 200, which ventilates a patient 300. An electromyographic signal is detected at the respiratory muscles (diaphragm and auxiliary muscles) of the patient via sensors and an original signal EMG(t) is sent to a signal processing unit 310, which determines an enveloping curve −EMG(t) from the original signal. Another signal processing unit 320 then determines a curve of the respiratory muscle pressure Pmus of the patient 300 from the enveloping curve −EMG(t) and from the signals provided by the ventilation device 200 (airway pressure, Paw(t), volume flow V′(t) and tidal volume V(t)).

Accordingly, the determination of the first information 12 and of the determination of the second information 14 may comprise an estimation (representation) of the breathing pressure generated by the muscles of the patient on the basis of an airway pressure generated at the patient Paw, of a tidal volume V and of a tidal volume flow V′. The process 10 may comprise a receipt of information on the generated airway pressure Paw, the tidal volume V and the tidal volume flow V′ from a ventilation system 200 ventilating the patient.

The iPEEP is the pressure that the lungs have at the end of a breath (end of the exhalation). When iPEEP is excessively high, the patient is not able to breathe sufficient air into the lungs against the iPEEP. Therefore, the knowledge of the iPEEP is important for diagnoses and for the control of a ventilator. Exemplary embodiments make it possible to calculate the iPEEP, without inserting a catheter into the patient and a measuring probe into the esophagus, i.e., without knowledge of the esophageal pressure Pes. Pvent designates below the pressure generated by the ventilator; Vol, Vol′ designates the total volume or the total volume flow, which flows from the ventilator to the patient or vice versa; and Volmus, Vol′mus designate the volume and the volume flow, respectively, which the patient achieves with his respiratory muscles.

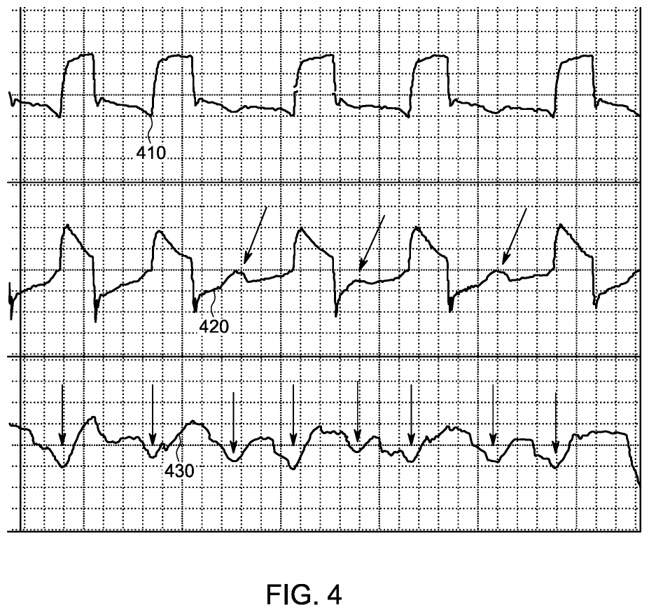

FIG. 4 shows a time course of a pressure generated by a ventilator Pvent, 410, a volume flow 420 between the ventilator and the patient, Vol′, and a curve of intrinsic breathing efforts 430 of a patient Pes. The ventilator operates in this exemplary embodiment in a pressure-controlled manner, i.e., the flow becomes established, and the pressure is predefined. As an alternative, it would also be possible to carry out the control the other way around, but this is rather not made use of during spontaneous breathing, but only in case of mandatory ventilation. The lower curve 430 shows the intrinsic breathing efforts of the patient, measured as esophageal pressure Pes. It can be seen that the spontaneous respiration rate is twice as high as the frequency of the ventilation strokes. The ventilator is not synchronized optimally with the intrinsic breathing activity of the patient.

Exemplary embodiments may use the curve of the respiratory muscle pressure here in order to carry out, for example, an automatic detection of times, at which the patient begins an attempt to inhale.

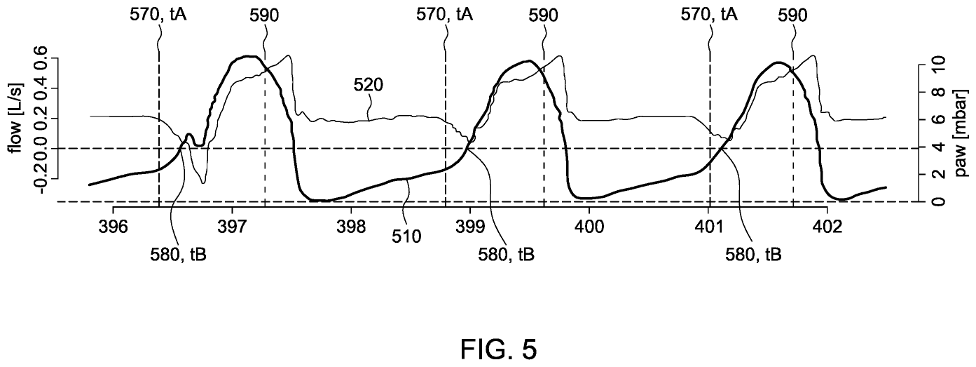

FIG. 5 shows a time course of a volume flow Vol′ 510 and a time course of an airway pressure Paw 520 in an exemplary embodiment. In the time course 510 of the volume flow Vol′ in [Usec], segments above the zero line designate an inhalation process. Paw is the time course 520 of the pressure Pvent in [mbar], which the ventilator achieves, and which is always greater than or equal to zero. The vertical broken lines 570 show the times of the beginning of the breathing effort, and the lines 590 show the times of the end of the breathing effort. The patient has COPD (chronic obstructive pulmonary disease) and suffers from “dynamic hyperinflation” or intrinsic PEEP. This is recognized from the fact that the Paw signal 520 drops long before the flow 510 has its zero crossing. The patient consequently must work hard to bring the flow 510 to zero from negative values (exhalation). A p0.1 occlusion is triggered at the time t=396.5 sec. Each vertical line 570 shows the beginning of an attempt of the patient to inhale. This time is designated by tA. The determination 12 of the first information comprises here an estimation (representation) of the first time, tA, on the basis of a start of a breathing gas flow in the direction of the patient generated by the muscles of the patient. The determination 12 of the first information may be carried out by estimating the first time, tA, on the basis of a breathing effort signal of the patient passing through a threshold value. The first time, tA, may likewise be estimated on the basis of a starting of a spontaneous breathing of the patient Each zero crossing of Vol′ 580 shows a time, from which the airway flow flows in the direction of the patient. This still does not correspond to an active supply. For this, the ventilator must first still trigger, which normally happens shortly thereafter, so that a pressure assistance stroke is then triggered. This time is designated by tB. The determination 12 of the second information may comprise an estimation of the second time, tB, on the basis of a start of a breathing gas flow in the direction of the patient. Because the ventilation strokes of the ventilator lag behind, cf. curve of the total volume flow 510, tB is behind tA in terms of time. The time course of Pmus can be calculated by means of sEMG. Accordingly, the determination 12, 14 of the first information and of the second information may comprise an estimation of the breathing pressure generated by the muscles of the patient on the basis of an electromyographic signal.

The determination 16 may comprise in exemplary embodiments a determination of an indicator of a difference or a weighted difference between Pmus (tA) and Pmus (tB). For example,

iPEEP=Pmus(tB)−Pmus(tA) (3)

is obtained in one exemplary embodiment. The individual summands may also be weighted here. In further exemplary embodiments, the determination 16 may comprise a determination of an indicator of a quotient or a weighted quotient between Pmus (tA) and Pmus (tB). The determination 16 may comprise an averaging, a sorting and/or an assessment of measured pressure values from a plurality of breathing phases. For example, the difference from the above equation is calculated several times in sequence, and the mean value is then formed.

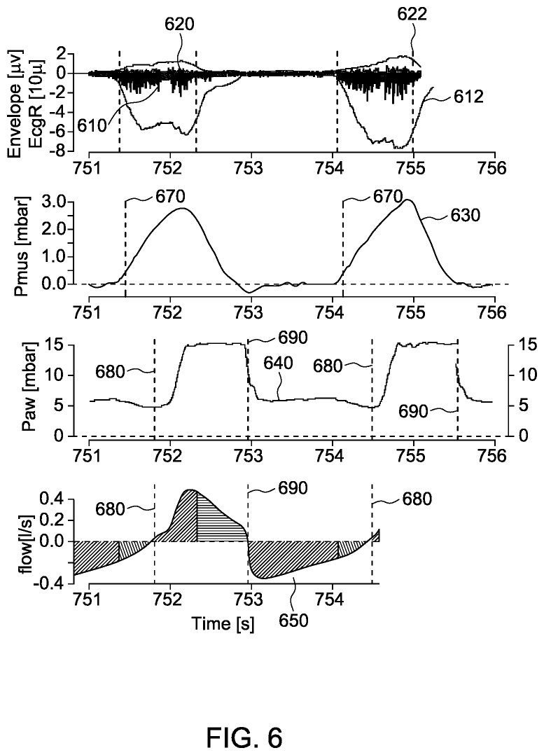

FIG. 6 shows signal curves of an EMG signal 610, 620, of a respiratory muscle pressure Pmus 630, of an airway pressure Paw 640 and of a volume flow Vol′ 650 in an exemplary embodiment. The topmost curve of FIG. 6 shows the time course of electrical signals 610, 620, which were generated by sEMG. Costmar 620 shows the time course, which was obtained on the basis of two measuring electrodes close to the diaphragm, Intercost 610 shows the time course, which was obtained on the basis of two electrodes at the ribs (auxiliary respiratory muscles), cf. also FIG. 3. In the two raw signals, the heartbeat time periods, which led to gaps, were removed by calculation (EcgR=ECG Removal). In the diagram shown the gaps were interpolated. The two corresponding solid lines 612, 622 (curves, envelopes) are an indicator of the spontaneous breathing of the patient. Pmus 630 is estimated on the basis of the formula (1) Pmus=Ecw·Vol−Pes. Each vertical line 670 is a time tA (Pmus>threshold value), i.e., the beginning of an attempt of the patient to inhale. As an alternative or in addition, Pmus may also be estimated as a function of Costmar 620 and Intercost 610, for example, as described in DE 10 2020 133 460 A1 (which is incorporated herein by reference). Pmus 630 must, in general, be estimated as a function of Costmar 620 and Intercost 610. For example, Pmus 630 can be calculated as a linear combination (with the coefficients NME 1 and NME 2) from Costmar 620 and Intercost 610.

The third curve 640 shows the airway pressure Paw. The fourth curve 650 shows the breathing gas flow Vol′ and illustrates that the ventilation strokes are not synchronized optimally with the intrinsic breathing effort of the patient. The vertical lines 680 designate the starting of an actual flow in the direction of the patient (beginning of inhalation phase, tB), and the lines 690 denote the starting of an actual flow away from the patient (beginning of exhalation phase). The vertical lines 680, 690 indicate each a phase change in the respective signal and the phase change of the breathing gas pressure 640 and the phase change of the breathing gas volume flow 650 lag behind times tA, at which the patient attempts to inhale spontaneously. Phase changes denote here the breathing phase change (between inhalation and expiration), which the ventilator detects (somewhat later).

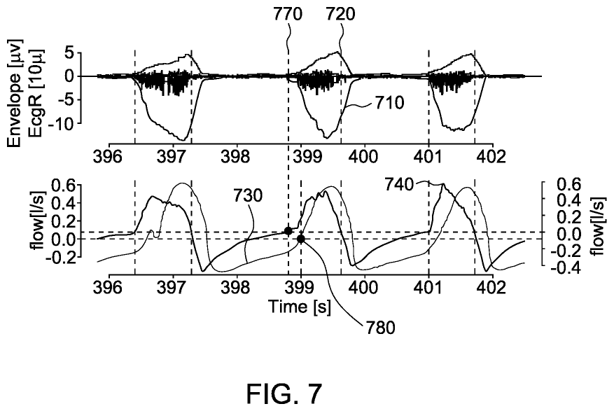

FIG. 7 shows signal curves of an sEMG signal 710, 720 of a volume flow Vol′ 730 and of a volume flow generated by the muscles of the patient, Vol′mus, 740 in an exemplary embodiment. FIG. 7 shows a typical situation, when the patient has COPD and therefore a higher iPEEP develops: Vol′ 730 denotes the total volume flow between the ventilator and the patient, and Vol′mus=Flow−mus=fmus 740 denotes the component of the total volume flow generated by the patient. The vertical lines 770 indicate when the patient is beginning the attempt to inhale. The zero crossing 780 of the total volume flow Vol′ 730 is always behind a line 770 in terms of time. An occlusion with 100 msec duration is carried out at the time t=396.5.

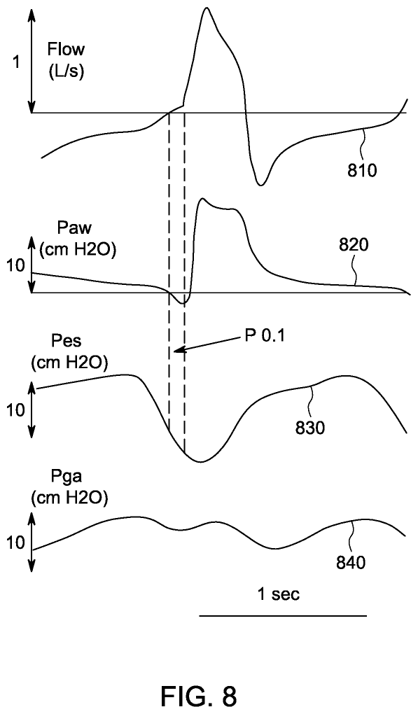

FIG. 8 shows signal curves of a volume flow 810, of an airway pressure Paw 820, of an esophageal pressure Pes 830 and of a gastric pressure Pga 840. During detection of the esophageal pressure Pes, the gastric pressure is frequently also detected via a second lumen of the esophageal catheter. The difference between the esophageal pressure and the gastric pressure yields an additional indicator of the muscle pressure that the diaphragm exerts. Here, Flow 810 means the volume flow generated, i.e., Vol′, Paw 820 means the pressure present at the ventilator, i.e., Paw, and P0.1 means an occlusion, which is triggered by a zero crossing of − to + of Vol′ 810, i.e., at a time tB, and lasts, for example, Δt=100 msec. Pes has already become lower at the time tA. In some exemplary embodiments, an occlusion may be triggered at a in plurality of times tB. The process 10 may accordingly comprise an averaging, a smoothing, a suppression of outliers or a determination of a median of a plurality of indicator of the iPEEP determined sequentially in time in order to obtain an improved indicator of the iPEEP. The ventilator measures the signal Paw 820 for the airway pressure. During the occlusion, i.e., in the time window of 100 msec duration, this signal Paw, except for the sign, coincides well with the pressure Pmus, which is generated by the intrinsic breathing effort of the patient. In the time window between tA and tB+Δt, moreover, Pmus may be assumed to be rising linearly. Pmus thus increases linearly from the value Pmus(tB) to the value Pmus(tB+Δt).

Thus, the following are valid:

iPEEP=Pmus(tB)−Pmus(tA), see above (3)

as well as

P0.1=Paw(tB)−Paw(tB−Δt), (4)

which is the pressure difference measured during the occlusion.

Since the Pmus curve in the interval [tA, tB+Δt] is considered to be linear, the following is valid:

) [Pmus(tB)−Pmus(tA)]/[tB−tA]=[Paw(tB)−Paw(tB+Δt)]/Δt. (5

From this and from (3) follows

iPEEP=[Paw(tB)−Paw(tB+Δt)][tB−tA]/Δt. (6)

In exemplary embodiments, the occlusion duration Δt may have, for example, a value of about 100 msec. In a generalization of (3),

iPEEP=f(ΔPmus) with ΔPmus=Pmus(tB)−Pmus(tA). (7)

For example, f(x)=a·x+b with an increase a and with an offset b.
From the equations (6) and (7) follows

a·[Pmus(tB)−Pmus(tA)]+b=[Paw(tB)−Paw(tB+Δt)]·[tB−tA]/Δt. (8)

A plurality of occlusions yield a sample, and a linear regression analysis yields estimations for the two parameters a and b.

In FIG. 8, the p01 occlusion is triggered after passing through the flow zero line. However, the breathing effort has already begun beforehand. This can be detected at the drop of the esophageal pressure Pes 830, which corresponds to the drop in Pmus (since the volume in the breath at the time is negligible). Furthermore, the Pmus signal used can be calibrated by interspersion of conventional and clinically accepted p0.1 occlusions, cf. FIG. 8 and equation (8). Therefore, the process 10 may comprise a calibration of the determination 16 of the indicator of the iPEEP on the basis of a measurement of a ventilation device 200 during an occlusion. The duration of these occlusions is about 100 msec, so that they are hardly noticed by the patient themself. The interspersion may take place at regular intervals, be initiated by the physician or be triggered as a function of properties, which concern the signal quality of the Pmus signal and of the sEMG signal, as is described, for example, in DE 10 2019 007 717 B3 (which is incorporated herein by reference). The pressure signal Paw detected by the ventilator 200 during the p0.1 occlusion is an almost ideal indicator of the muscle pressure Pmus in this small (100 msec) time window. Furthermore, it can be expected that the increase in the Pmus is constant around the time tA, i.e., the increase of Pmus during the interval [tA, tB] corresponds to the increase during the occlusion, i.e.:

ΔPmus/(tB−tA)=(Pmus(tB)−Pmus(tA))/(tB−tA)=iPEEP/(tB−tA)=p0.1/100 msec, (9)

and

iPEEP=p0.1·(tB−tA)/100 msec, (10)

cf. also equation (6).

The Pmus signal or directly the value iPEEP can be checked and possibly scaled/calibrated by means of this continuity condition by using p0.1 occlusions (see below, equations (11) and (12)). By the way, this equation (10) is also used in the context of ultrasound measurement, as is known from Bernardi E. et al.: “A New Ultrasound Method for Estimating Dynamic Intrinsic Positive Airway Pressure: A Prospective Clinical Trial,” AJRCCM, as well as from Pisani L. et al.: “Noninvasive Detection of Positive End-expiratory Pressure in COPD Patients Recovering from Acute Respiratory Failure,” European Respiratory Journal 2016. The calculation of the dynamic intrinsic PEEP does not absolutely have to be carried out as shown in equation (2), since correction terms are possibly necessary (for this, see discussion in Younes, M.: “Dynamic Intrinsic PEEP (PEEPi,dyn) Is It Worth Saving,” AJRCCM).

However, it is assumed that

iPEEP=f(Pmus)=f(Pmus(tB)−Pmus(tA)) (11)

is valid, wherein the function f(x) is preferably a linear function

f(x)=a·x+b. (12)

The factor and the offset b may be determined, for example, from a regression (calibration) by means of p0.1 occlusions. The regression equation is obtained after equating iPEEP from equations (10) and (11)

p0.1·(tB−tA)/100 msec=a·ΔPmus+b, (13)

cf. equation (8).

Because of the uncomplicated repeatability of the measurements of p0.1 and ΔPmus, a and b can be determined directly, for example, with linear regression. The process steps described above may be carried out in exemplary embodiments by the control unit 24 of the device 20. This control unit may be configured to carry out the determination 16 of the indicator of the iPEEP, which may comprise, for example, a determination of an indicator of a difference or a weighted difference between Pmus (tA) and Pmus (tB), a determination of an indicator of a quotient or a weighted quotient between Pmus (tA) and Pmus (tB) or an averaging, a sorting and/or an assessment of measured pressure values from a plurality of breathing phases.

The control unit 24 may be configured to carry out the determination 12, 14 of the first information and of the second information by an estimation of the breathing pressure generated by the muscles of the patient on the basis of an electromyographic signal and/or by an estimation of the breathing pressure generated by the patient on the basis of an airway pressure generated at the patient Paw, of a tidal volume V and of a tidal volume flow V′.

The control unit 24 may be configured to receive, for example, via the one or more interfaces 22, information on the airway pressure generated Paw, the tidal volume V and the tidal volume flow V from the ventilation device 200 ventilating the patient 300.

The control unit 24 may be configured in some exemplary embodiments to determine 12 the first information by an estimation of the first time, tA, on the basis of a starting of a breathing gas flow in the direction of the patient generated by the muscles of the patient, by an estimation of the first time, tA, on the basis of a breathing effort signal of the patient passing through a threshold value and/or by an estimation of the first time, tA, on the basis of a starting of a spontaneous breathing of the patient.

The control unit 24 may be configured in further exemplary embodiments to determine the second information by an estimation of the time, tB, on the basis of a starting of a breathing gas flow in the direction of the patient. The control unit 24 may be configured to average a plurality of indicators of the iPEEP determined sequentially in time, to smooth them, to suppress outliers or to determine a median from them, in order to obtain an improved indicator of the iPEEP. The control unit 24 may be configured to calibrate the determination 16 of the indicator of the iPEEP on the basis of a measurement of the ventilation device during one or more occlusions. For example, the above-described p0.1 occlusions are used for this since they are of short duration and are available as a function in many ventilators.

The aspects and features that are described in connection with one or more of the examples and figures described in detail above may also be combined with one or more of the other examples in order to replace an identical feature of the other example or in order to additionally introduce the feature into the other example.

Examples may, furthermore, be or pertain to a computer program with a program code for executing one or more of the above processes when the computer program is executed on a computer or on a processor. Steps, operations or processes of different processes described above may be executed by programmed computers or processors. Examples may also cover program memory devices, e.g., digital storage media, which are machine-readable, processor-readable or computer-readable and code machine-executable, processor-executable or computer-executable programs of instructions. The instructions execute some or all of the steps of the above-described processes or cause them to be executed. The program memory devices may comprise or be, for example, digital memories, magnetic storage media, for example, magnetic disks and magnetic tapes, hard drives or optically readable digital storage media. Further examples may also cover computers, processors or control units, which are programmed to execute the steps of the above-described processes, or (field)-programmable logic arrays ((F)PLAs=(Field) Programmable Logic Arrays) or (field)-programmable gate arrays ((F)PGA=(Field) Programmable Gate Arrays), which are programmed to execute the steps of the above-described processes.

Only the principles of the disclosure are represented by the description and the drawings. Furthermore, all the examples mentioned here shall be used, in principle, expressly only for purposes of illustration in order to support the reader in understanding the principles of the disclosure and of the concepts contributed by the inventor (inventors) to the further development of the technology. All the statements made here about principles, aspects and examples of the disclosure as well as concrete examples thereof comprise equivalents thereof.

A function block designated as “means for . . . ” performing a certain function may pertain to a circuit, which is configured for performing a certain function. Thus, a “means for something” may be implemented as a “means configured for something or suitable for something,” e.g., a structural component or a circuit configured for or suitable for the respective task.

Functions of different elements shown in the figures, including those of each function block designated as “means,” means for providing a signal,” “means for generating a signal,” etc., may be implemented in the form of dedicated hardware, e.g., “a signal provider,” “a signal processing unit,” “a processor,” “a control,” etc., as well as hardware capable of executing software in connection with corresponding software. In case of provision by a processor, the functions may be provided by an individual dedicated processor, by an individual, jointly used processor or by a plurality of individual processors, some of which or all of which may be used jointly. However, the term “processor” or “control” is far from being limited to hardware capable exclusively of executing software, but it may comprise digital signal processor hardware (DSP hardware; DSP=Digital Signal Processor), network processor, application-specific integrated circuit (ASIC=Application Specific Integrated Circuit), field-programmable logic array (FPGA=Field Programmable Gate Array), read-only memory (ROM:=Read Only Memory) for storing software, random access memory (RAM=Random Access Memory) and non-volatile storage device (storage). Other hardware, conventional and/or customer-specific, may be included as well.

A block diagram may represent, for example, a schematic circuit diagram, which implements the principles of the disclosure. Similarly, a flow chart, a flow diagram, a state transition diagram, a pseudocode and the like may represent different processes, operations or steps, which are represented, for example, essentially in computer-readable medium and are thus executed by a computer or processor, regardless of whether such a computer or processor is explicitly shown. Processes disclosed in the description or in the patent claims may be implemented by a component, which has means for carrying out each and every one of the respective steps of these processes.

It is apparent that the disclosure of a plurality of steps, processes, operations or functions disclosed in the description or in the claims shall not be implemented as being arranged in the defined order, unless this is explicitly or implicitly stated otherwise, e.g., for technical reasons. Therefore, these are not limited by the disclosure of a plurality of steps or functions to a defined order, unless these steps or functions are not replaceable for technical reasons. Further, an individual step, function, process or operation may include in some examples a plurality of partial steps, partial functions, partial processes or partial operations and/or be broken up into these. Such partial steps may be included and be a part of the disclosure of this individual step, unless they are explicitly excluded.

Furthermore, the following embodiments are hereby included in the detailed description, in which each embodiment can stand by itself as a separate example. While each embodiment can stand by itself as a separate example, it should be noted that, even though embodiments that reference other embodiments may pertain in the embodiment to a defined combination with one or more other embodiments, other examples may also comprise a combination of the referencing embodiment with the subject of every other the referencing embodiment or the non-referencing embodiment. Such combinations are explicitly proposed here, unless it is stated that a defined combination is not intended. Furthermore, features of a referencing embodiment shall also be included for every other non-referencing embodiment, even if this referencing embodiment does not explicitly reference the other non-referencing embodiment.

Further and preferred embodiments of the present invention will be described in more detail below with respect to a concept for determining an indicator of an intrinsic end-expiratory pressure (iPEEP) in the lungs of a patient on the basis of an analysis of a time course of a breathing pressure generated by the muscles of the patient.

A basic embodiment includes a device for a ventilation device and for determining an indicator of an intrinsic end-expiratory pressure, iPEEP, in the lungs of a patient, with one or more interfaces, which are configured for the exchange of information with the ventilation device, and with a control unit, which is configured
to determine a first information on a first breathing pressure generated by the muscles of the patient, Pmus (tA), at a first time, tA, at which an inhalation attempt of the patient is present;
to determine a second information on a second breathing pressure generated by the muscles of the patient, Pmus (tB), at a second time, tB, at which a breathing gas flow towards the patient starts; and
to determine the indicator of the iPEEP on the basis of the first information and on the basis of the second information.

A preferred embodiment based on the above-described embodiments may comprise one or more sensors for the detection of measured values during a ventilation of a patient.

A preferred embodiment based on one of the above-described embodiments may be configured to detect measured pressure values or measured pressure signals during a ventilation of a patient.

In a preferred embodiment based on at least one of the above-described embodiments, the determination may comprise a determination of an indicator of a difference or a weighted difference between Pmus (tA) and Pmus (tB).

In a preferred embodiment based on at least one of the above-described embodiments, the determination may comprise a determination of an indicator of a quotient or a weighted quotient between Pmus (tA) and Pmus (tB).

In a preferred embodiment based on at least one of the above-mentioned embodiments, the determination may comprise an averaging, a sorting and/or an assessment of measured pressure values from a plurality of breathing phases.

In a preferred embodiment based on at least one of the above-mentioned embodiments, the determination of the first information and of the piece of information may comprise an estimation of the breathing pressure generated by the muscles of the patient on the basis of an electromyographic signal.

In a preferred embodiment based on at least one of the above-described embodiments, the determination of the first information and of the second information may comprise an estimation of the breathing pressure generated by the muscles of the patient on the basis of an airway pressure generated at the patient Paw, of a tidal volume V and of a tidal volume flow V′.

In a preferred embodiment based on at least one of the above-described embodiments, the control unit may be configured to receive a piece of information on the airway pressure generated Paw, of the tidal volume V and the tidal volume flow V′ from the ventilation device ventilating the patient.

In a preferred embodiment based on at least one of the above-described embodiments, the determination of the piece of information may comprise an estimation of the first time on the basis of a starting of a breathing gas flow generated by the muscles of the patient in the direction of the patient.

In a preferred embodiment based on at least one of the above-described embodiments, the determination of the first information may comprise an estimation of the first time on the basis of a starting of a spontaneous breathing of the patient.

In a preferred embodiment based on at least one of the above-described embodiments, the determination of the second information may comprise an estimation of the second time on the basis of a starting of a breathing gas flow in the direction of the patient.

in a preferred embodiment based on at least one of the above-described embodiments, the control unit may be configured to average a plurality of indicators of the iPEEP determined sequentially in time, to smooth them, to suppress outliers or to determine a median therefrom in order to obtain an improved indicator of the iPEEP.

In a preferred embodiment based on the above-described embodiments, the control unit may be configured to calibrate the determination of the indicator of the iPEEP on the basis of a measurement of the ventilation device during an occlusion.

In a preferred embodiment based on at least one of the above-described embodiments, the device may be configured as a ventilation device.

A basic embodiment includes a process for determining an indicator of an intrinsic end-expiratory pressure, iPEEP, in the lungs of a patient, with
determination of a first information on a first breathing pressure generated by the muscles of the patient, Pmus (tA), at a first time tA, at which an inhalation attempt of the patient is present;
determination of a second information on a second breathing pressure generated by the muscles of the patient, Pmus (tB), at a second time, tB, at which a breathing gas flow towards the patient starts,
determination (16) of the indicator of the iPEEP based on the first information and on the basis of the second information.

In a preferred embodiment based on the above-described embodiment, the determination may comprise a determination of an indicator of a difference or a weighted difference between Pmus (tA) and Pmus (tB).

In a preferred embodiment based on at least one of the above-described embodiments, the determination may comprise a determination of an indicator of a quotient or a weighted quotient between Pmus (tA) and Pmus (tB).

In a preferred embodiment based on at least one of the above-described embodiments, the determination may comprise an averaging, a sorting and/or an assessment of measured pressure values from a plurality of breathing phases.

In a preferred embodiment based on at least one of the above-described embodiments, the determination of the first information and of the second information may comprise an estimation of the breathing pressure generated by the muscles of the patient on the basis of an electromyographic signal.

In a preferred embodiment based on at least one of the above-described embodiments, the determination of the first information and of the second information may comprise an estimation of the breathing pressure generated by the muscles of the patient on the basis of an airway pressure generated at the patient Paw, of a tidal volume V and of a tidal volume flow V′.

A preferred embodiment based on at least one of the above-described embodiments may comprise a receipt of information on the airway pressure generated Paw, the tidal volume V and the tidal volume flow V′ from a ventilation system ventilating the patient.

In a preferred embodiment based on at least one of the above-described embodiments, the determination of the first information may comprise an estimation of the first time, tA, on the basis of a starting of a breathing gas flow in the direction of the patient generated by the muscles of the patient.

In a preferred embodiment based on at least one of the above-described embodiments, the determination of the first information may comprise an estimation of the second time, tB, on the basis of a starting of a breathing gas flow in the direction of the patient.

A preferred embodiment based on at least one of the above-described embodiments may comprise an averaging, smoothing, suppression of outliers or determination of a median of a plurality of indicators of the iPEEP determined sequentially in time in order to obtain an improved indicator of the iPEEP.

A preferred embodiment based on at least one of the above-described embodiments may comprise a calibration of the determination of the indicator of the iPEEP on the basis of a measurement of a ventilation device during an occlusion.

A basic embodiment may comprise a computer program with a program code for executing at least one of the above-described embodiments. The program code may advantageously be executed on a computer, on a processor or on a programmable hardware component.

Table 2 below comprises the abbreviations and terms used within the framework of the present invention along with respective brief explanations.

TABLE 2

| Symbol | Description/Brief explanation | Units |
|---|---|---|
| R | Resistance of the respiratory system; R = P/(dV/dt) | mbar/(L/sec) |
| C | Compliance; C = V/P | L/mbar |
| E | Elasticity of the respiratory system; E = 1/C; E = P/V | mbar/L |
| τ, tau | Time constant of the respiratory system | sec |
| s | Frequency (complex number) | s⁻¹ |
| Flow, dV/dt | Airway gas flow, flow rate of gases, which flows away from | L/min, L/sec |
| | the patient or flows towards the patient | |
| Vol, V | Volume, which flows from/to the patient | L |
| sEMG, | EMG signal, time course of the EMG signal | μV |
| sEMG(t) | | |
| MV | Minute volume | L |
| V_{T} | Tidal volume of the patient | L |
| Vol′_{ges} | Total volume flow, which flows from/to the patient. | L/min, L/sec |
| Volₘᵤₛ | Volume caused by muscle activity | L |
| Vol_{musMax} | Maximum volume caused by muscle activity | L |
| Vol′ₘᵤₛ | Volume flow caused by muscle activity | L/sec |
| Flowₛₚₒₙ | Breathing gas flow caused by spontaneous muscle activity | L/min, L/sec |
| Flowₘᵤₛ | Volume flow caused by muscle activity | L/min, L/sec |
| Volᵥₑₙₜ | Volume produced by the ventilator | L |
| Vol′ᵥₑₙₜ | Breathing gas flow produced by the ventilator | L/min, L/sec |
| Volₛₜᵢₘ | Volume produced by stimulation | L |
| V_{T mus} | Tidal volume produced by muscle activity | L |
| M_{Vmus} | Minute volume produced by muscle activity | L |
| P_{aw} | Airway pressure | mbar |
| P_{drv} | Total pressure acting on the respiratory system (driving | mbar |
| | pressure) | |
| Pᵥₑₙₜ | Ventilation pressure, the pressure generated by the ventilator | mbar |
| Pₘᵤₛ, Pₘᵤₛ(t) | Pressure caused by muscle activity | mbar |
| P_{mus base}, | Basic breathing load, the minimum pressure that is necessary | mbar |
| P_{base} | to overcome the respiratory resistances | |
| P_{musMax} | Pressure caused by maximum muscle activity | mbar |
| PIₘₐₓ | Maximum pressure generated during inhalation with the | mbar |
| | mouth closed | |
| Pₛₚₒₙ | Spontaneous muscle pressure, pressure caused by | mbar |
| | spontaneous muscle activity | |
| Pₛₜᵢₘ | Stimulated muscle pressure, pressure caused by muscle | mbar |
| | stimulation | |
| Pᵢₙₛₚ | Airway pressure during the phase of inhalation (inspiration) | mbar |
| P_{exp.} | Airway pressure during the phase of exhalation (expiration) | mbar |
| PEEP | Positive end-expiratory pressure at the end of the phase of | mbar |
| | exhalation | |
| iPEEP | Intrinsic end-expiratory pressure | mbar |
| WOB | Work of Breathing; | Nm, J |
| | [mbar * L] = [IhPa * m³/1000] = 0.1 Nm | |
| WOBₜₒₜ | Total, work of breathing | Nm, J |
| WOBᵥₑₙₜ | Work of breathing produced by the ventilator | Nm, J |
| WOBₛₚₒₙ | Work of breathing produced by spontaneous musele activity | Nm, J |
| WOBₛₜᵢₘ | Work of breathing produced by stimulation | Nm, J |
| WOB_{base} | Basic breathing load in the sense of work of breathing | Nm. J |
| WOB_{musMax} | Work of breathing produced by maximum muscle activity | Nm, J |
| NVE | Neuroventilatory efficiency | L/μV |
| NME | Neuromechanical efficiency | mbar/V |
| LBC | Load Bearing Capacity | — |
| LI | Load Index | — |
| EMGₛₜᵢₘ | EMG signal brought about by stimulation | μV |
| EMGₛₚₒₙ | EMG signal brought about by spontaneous activity | μV |
| SVE(t) | Ventilatory efficiency relative to muscle stimulation. | L/% |
| | SVE(t) = k(t) · NVEstim | |
| k(t) | Activatability | μV/% |
| NVEₛₜᵢₘ | Neuroventilatory efficiency during stimulation | L/μV |
| NMEₛₜᵢₘ | Neuromechanical efficiency during stimulation | mbar/μV |
| NMEₛₚₒₙ | Neuromechanical efficiency during spontaneous breathing/ | mbar/μV |
| | spontaneous muscle activity | |
| NVEₛₚₒₙ | Neuroventilatory efficiency during stimulation breathing/ | L/μV |
| | spontaneous muscle activity | |
| Iₛₜᵢₘ, Iₛₜᵢₘ(t) | Stimulation intensity, intensity of the stimulation sign | — |
| a2 | Component of driving pressure | — |
| b2 | Component of driving pressure | — |
| α | Positive feedback factor | — |
| beta | β = α/(1 + α) | — |

All the patent documents and publications along with publication numbers and with short titles of the publications are listed in Table 3 below. The full titles can be found in the explanations on the state of the art in the introduction to the specification. The reference numbers [E1] through [E38] listed in this table are used at times in the specification.

| [Ref] | Publication/disclosure/brief title |
|---|---|
| [E1] | U.S. Pat. No. 5,820,560 |
| [E5] | WO2019154834 |
| [E6] | WO2019154837 |
| [E7] | WO2019154839 |
| [E8] | WO2020079266 |
| [E9] | DE102019006480 |
| [E11] | Walker, D. J.: “Prediction of the esophageal pressure . . .” |
| (E13] | US2017/0252558 |
| [E14] | DE102019006480 |
| [E15] | DE102019007717 |
| [E16) | US2021205561 (A1) (DE102020000014) |
| [E17] | DE102007062214 |
| [E18] | WO2018143844 |
| [E19] | Kahl, L. et al.: “Comparison of algorithms to . . .” |
| [E20] | DE102015011390 |
| [E21] | Jansen D. et al.: “Estimation of the diaphragm neuromuscular |
| | . . .” |
| [E22] | Liu L. et al.: “Neuroventilatory efficiency and extubation |
| | readiness . . .” |
| [E23] | Cattapan, S. E. et al.: “Can diaphragmatic contractility be |
| | assessed by . . .” |
| [E24] | Younes, M. et al.: “A method for monitoring and improving . . .” |
| [E25] | Putensen C. et al.: “Long-term effects of spontaneous breathing |
| | . . . \| |
| [E26] | Bellani, G. et al.:” “The Raio of Inspiratory Pressure Over . . .” |
| [E27] | U.S. Pat. No. 7,021,310 |
| [E28] | US20120103334 |
| [E29] | DE102021115865 |
| [E30] | Otis, A. B. et al.: “Mechanics of Breathing in Man” |
| [E31] | US20150366480 |
| [E32] | WO2020188069 |
| [E34] | US 2022 072249 (A1) (DE102020123138) |
| [E35] | Shalaby, R. E.-S.: “Development of an Electromyography |
| | Detection System . . .” |
| [E36] | Osuagwu, B. A. C, et al.: “Active proportional electromyogram |
| | controlled . . .” |
| [E37] | US2009114224(A1)(DE102007052897) |
| [E38] | Virtanen, J. et al.: “Instrumentation for the measurement of |
| | electric . . .” |

While specific embodiments of the invention have been shown and described in detail to illustrate the application of the principles of the invention, it will be understood that the invention may be embodied otherwise without departing from such principles.

List of Reference Numbers

| | |
|---|---|
| 10 | Process for determining an indicator of an intrinsic end- |
| | expiratory pressure, iPEEP |
| 12 | Determination of a first information |
| 14 | Determination of a second information |
| 16 | Determination of the indicator of the iPEEP |
| 20 | Device for determining an indicator of an intrinsic end-expiratory |
| | pressure, iPEEP |
| 22 | One or more interfaces |
| 24 | Control unit |
| 200 | Ventil ation device |
| 300 | Patient |
| 310 | Signal processing |
| 320 | Signal processing |
| 410 | Pvent, pressure generated by a ventilator |
| 420 | Vol′, volume flow |
| 430 | Pes, curve of intrinsic breathing efforts |
| 510 | Volume flow, Vol′ in [L/sec] |
| 520 | Paw, time course of the pressure Pvent in [mbar] |
| 570 | Times of the beginning of the breathing effort, tA |
| 580 | Zero crossing of Vol′, tB |
| 590 | End of the breathing effort |
| 610 | Intercost |
| 612 | Envelope of Intercost |
| 620 | Costmar |
| 630 | Respiratory muscle pressure, Pmus |
| 640 | Airway pressure, Paw |
| 650 | Volume flow; Vol′ |
| 680 | Start of an actual flow in the direction of the patient, tB |
| 690 | Start of an actual flow away from the patient |
| 710 | sEMG signal, Intercost |
| 720 | SEMG signal. Costmar |
| 730 | Volume flow, Vol′ |
| 740 | Muscle-generated volume flow, Vol′mus |
| 770 | Inhalation attempt, tA |
| 780 | Zero crossing of the total volume flow Vol′, tB |
| 810 | Volume flow, Flow |
| 820 | Airway pressure, Paw |
| 830 | Esophageal pressure, Pes |
| 840 | Gastric pressure, Pga |

What is claimed is:

1. A device for determining an indicator of an intrinsic end-expiratory pressure in lungs of a patient, the device comprising:
an interface configuration comprising one or more interfaces configured for an exchange of information with a ventilation device; and
a control unit configured:
to determine first information on a first breathing pressure generated by muscles of the patient, at a first time, at which an inhalation attempt of the patient is present;
to determine second information on a second breathing pressure generated by the muscles of the patient, at a second time, at which breathing gas flow towards the patient starts; and
to determine the indicator of the intrinsic end-expiratory pressure based on the first information and based on the second information.

2. A device in accordance with claim 1, further comprising a sensor arrangement comprising one or more sensors for detecting measured values during a ventilation of a patient.

3. A device in accordance with claim 1, wherein the control unit is configured to detect measured pressure values or measured pressure signals during a ventilation of the patient.

4. A device in accordance with claim 1, wherein the determination of the indicator comprises determining one or more of:
a difference or a weighted difference between breathing pressure caused by muscle activity of the patient at the first time and breathing pressure caused by muscle activity of the patient at the second time; and
a quotient or a weighted quotient of breathing pressure generated by muscle activity of the patient at the first time and breathing pressure caused by muscle activity of the patient at the second time.

5. A device in accordance with claim 1, wherein the determination of the indicator comprises one or more of:
an averaging of measured pressure values from a plurality of breathing phases;
a sorting of measured pressure values from a plurality of breathing phases;
an assessment of measured pressure values from a plurality of breathing phases.

6. A device in accordance with claim 1, wherein the determination of the first information and of the second information comprises one or more of:
an estimation of the breathing pressure generated by muscle activity of the patient based on an electromyographic signal;
an estimation of the breathing pressure generated by muscle activity of the patient based on an airway pressure generated at the patient;
an estimation of the breathing pressure generated by muscle activity of the patient based on a tidal volume; and
an estimation of the breathing pressure generated by muscle activity of the patient based on a tidal volume flow.

7. A device in accordance with claim 1, wherein the control unit is configured to receive one or more of:
information on an airway pressure generated at the patient from a ventilation device currently ventilating the patient;
information on a tidal volume from a ventilation device currently ventilating the patient; and
information on a tidal volume flow from a ventilation device currently ventilating the patient.

8. A device in accordance with claim 1, wherein the determination of the first information comprises an estimation of the first time based on a starting of a breathing gas flow, in a direction of the patient, generated by the muscles of the patient.

9. A device in accordance with claim 1, wherein the determination of the first information comprises one or more of:
an estimation of the first time based on a patient breathing effort signal passing through a threshold value; and
an estimation of the first time based on a starting of a spontaneous breathing of the patient.

10. A device in accordance with claim 1, wherein the determination of the second information comprises an estimation of the second time based on a starting of a breathing gas flow in the direction of the patient.

11. A device in accordance with claim 1, wherein the control unit is configured to obtain an improved indicator of the indicator of the intrinsic end-expiratory pressure in lungs of the patient by one or more of:
averaging a plurality of indicators of the intrinsic end-expiratory pressure in lungs of the patient determined sequentially in time;
smoothing a plurality of indicators of the intrinsic end-expiratory pressure in lungs of the patient determined sequentially in time; and
suppressing outliers of a plurality of indicators of the intrinsic end-expiratory pressure in lungs of the patient determined sequentially in time; and
determining a median from a plurality of indicators of the intrinsic end-expiratory pressure in lungs of the patient determined sequentially in time.

12. A device in accordance with claim 1, wherein the control unit is configured to calibrate the determination of the indicator of the of indicator of the intrinsic end-expiratory pressure in lungs of the patient based on a measurement of the ventilation device during an occlusion.

13. A ventilation device comprising:
a device for determining an indicator of an intrinsic end-expiratory pressure in lungs of a patient, the device comprising:
an interface configuration comprising one or more interfaces configured for an exchange of information with a ventilation device; and
a control unit configured:
to determine first information on a first breathing pressure generated by muscles of the patient, at a first time, at which an inhalation attempt of the patient is present;
to determine second information on a second breathing pressure generated by the muscles of the patient, at a second time, at which breathing gas flow towards the patient starts; and
to determine the indicator of the intrinsic end-expiratory pressure based on the first information and based on the second information.

14. A ventilation device in accordance with claim 13, further comprising a sensor arrangement comprising one or more sensors for detecting measured values during a ventilation of a patient.

15. A ventilation device in accordance with claim 13, wherein the control unit is configured to detect measured pressure values or measured pressure signals during a ventilation of the patient.

16. A ventilation device in accordance with claim 13, wherein the determination of the indicator comprises determining one or more of:
a difference or a weighted difference between breathing pressure caused by muscle activity of the patient at the first time and breathing pressure caused by muscle activity of the patient at the second time; and
a quotient or a weighted quotient of breathing pressure generated by muscle activity of the patient at the first time and breathing pressure caused by muscle activity of the patient at the second time.

17. A ventilation device in accordance with claim 13, wherein the determination of the indicator comprises one or more of:
an averaging of measured pressure values from a plurality of breathing phases;
a sorting of measured pressure values from a plurality of breathing phases;
an assessment of measured pressure values from a plurality of breathing phases.

18. A ventilation device in accordance with claim 13, wherein the determination of the first information and of the second information comprises one or more of:
an estimation of the breathing pressure generated by muscle activity of the patient based on an electromyographic signal;
an estimation of the breathing pressure generated by muscle activity of the patient based on an airway pressure generated at the patient;
an estimation of the breathing pressure generated by muscle activity of the patient based on a tidal volume; and
an estimation of the breathing pressure generated by muscle activity of the patient based on a tidal volume flow.

19. A process for determining an indicator of an intrinsic end-expiratory pressure in the lungs of a patient, the process comprising the steps of:
determining first information on a first breathing pressure generated by muscle activity of the patient at a first time, at which an inhalation attempt of the patient is present;
determining second information on a second breathing pressure generated by muscle activity of the patient at a second time, at which a breathing gas flow towards the patient starts;
determining the indicator of the an intrinsic end-expiratory pressure based on the first information and based on the second information.

20. A process according to claim 19, wherein a computer program with a program code performs at least some of the process steps when the program code is executed on a computer, on a processor or on a programmable hardware component.
